(19) United States
(12) Patent Application Publication
(10) Pub. No.: US 2007/0004803 A1
(43) Pub. Date: Jan. 4, 2007

(54) Compounds and methods for use in treating neoplasia and cancer based upon inhibitors of isoprenylcysteine methyltransferase

(75) Inventors: Richard A. Gibbs, West Lafayette, IN (US); Brian S. Henriksen, San Diego, CA (US); Christine A. Hrycyna, Lafayette, IN (US); Jessica L. Anderson, West Lafayette, IN (US)

Correspondence Address: COLEMAN SUDOL SAPONE, P.C., 714 COLORADO AVENUE, BRIDGE PORT, CT, 06605-1601, US

(73) Assignee: Purdue Research Foundation, West Lafayette, IN (US)

(21) Appl. No.: 10/552,754

(22) PCT Filed: Mar. 26, 2004
(86) PCT No.: PCT/US04/09506

§ 371 Date: Aug. 11, 2006

Related U.S. Application Data
(60) Provisional application No. 60/457,862, filed on Mar. 26, 2003.

(51) Int. Cl. A61K 31/198; C07C 323/39

(52) U.S. Cl. 514/562; 554/42

(57) ABSTRACT

The present invention relates to a novel method for the treatment of neoplasia, including cancer and other diseases and conditions in humans and mammals. More particularly, in preferred aspects, the present invention provides a method for the use of prenylcysteine analogs for the treatment of neoplasia, hyperproliferative cell growth including psoriasis, restenosis following cardiovascular surgery, hyperplasia, including renal hyperplasia, chronic inflammatory diseases including rheumatoid and osteoarthritis, among others.

### FIELD OF THE INVENTION

The present invention relates to a novel method for the treatment of neoplasia, including cancer and other diseases and conditions in animals, including mammals, especially humans. More particularly, in preferred aspects, the present invention provides a method for the use of a novel class of chemical agents which are inhibitors of isoprenylcysteine methyltransferase, for the treatment of both neoplasia and cancer, and a number of hyperproliferative disorders, among others.

### BACKGROUND OF TH INVENTION

Cancer is a disease of abnormal cell growth often leading to death. Cancer is treated by three principal means; surgical removal of the tumor, therapeutic radiation, and treatment with anti-tumor chemical compounds. Treatment with chemical compounds, termed chemotherapy, is often hindered by the inherent toxicity of the chemicals to the patient and resistance of the tumor to the chemical treatment. Therefore the identification of less toxic anti-tumor agents capable of inhibiting growth of resistant tumors is of great importance.

Ras proteins and many other important signal transduction proteins must undergo significant post-translational modification in order to be functional in the eucaryotic cell. These proteins possess a signature carboxyl-terminal CaaX box motif (See FIG. 1), with is recognized by one of the two prenyltransferases, FTase (protein-farnesyltransferase) or GGTase I (protein-geranylgeranyltransferase I). FTase transfers the 15-carbon farnesyl moiety to the cysteine residue in certain CaaX sequences, while GGTase I transfers the 20-carbon geranylgeranyl moiety to different CaaX boxes. Ras proteins and certain other proteins are farnesylated, but the majority of naturally-occuring CaaX proteins are geranylgeranylated by GGTase I. Subsequent to prenylation, CaaX motif proteins are subjected to removal of the aaX residues by the protease RCE1, followed by SAM-dependent methylation of the resulting cysteine carboxylate by Icmt. These two membrane-bound enzymes recognize and modify both farnesylated and geranylgeranylated proteins. The overall result of these three post-translational steps is to convert a hydrophillic protein into a more hydrophobic, membrane-associated one.

The intense interest in this pathway, and specifically in FTase, was initially derived from the fact that mutant Ras proteins, the products of ras oncogenes, are key causative agents in ˜30% of human cancer. The development of selective inhibitors of FTase is a key area of current cancer chemotherapeutic research, and a large number of potent inhibitors of FTase have been developed, with two compounds in advanced trials for treatment of several carcinomas. Despite the promise demonstrated in the pre-clinical and clinical evaluation of these agents, they also exhibited a significant and surprising drawback: many human tumors driven by the mutant form of K-Ras are quite resistant to FTIs. It was expected that these tumors would be particularly sensitive to FTI treatment, since FTIs were designed to act as anti-Ras agents. However, it has been confirmed by several groups that, in the presence of FTIs, the crucial oncoprotein target K-Ras is geranylgeranylated by GGTase I, and this alternative modification apparently allows mutant K-Ras to continue its growth-promoting actions. Thus, there has been interest in developing other methods for the inactivation of Ras proteins.

There was significant early interest in these two steps catalyzed by RCE1 and Icmt, but progress in this area was stymied by an inability to isolate and purify these membrane-bound proteins. Moreover, it was felt that these steps were of secondary importance, as farnesylation itself seemed to be sufficient for activity of mutant Ras proteins. However, it has been recently demonstrated that genetic disruption of the mouse RCE1 or Icmt gene leads to a profound mislocalization of K-Ras, and thus presumably a blockage of its ability to promote cell growth. Taken together, these data may suggest that a) inhibition of Icmt might lead to mislocalization of both farnesylated and geranylgeranylated K-Ras; b) this mislocalization may well interefere with the biological activity of K-Ras, and thus c) Icmt inhibitors may be intriguing potential anticancer agents. The present application is thus directed to the examination of the substrate specificity of Icmt with a view toward the development of substrate-based inhibitors of the enzyme. In the present invention, active compounds are disclosed as anti-cancer/anti-tumor agents as well as agents to treat disease states or conditions which are modulated through isoprenyl cysteine methyltransferase enzyme, including hyperproliferative cell growth, restenosis following cardiovascular surgery, hyperplasia, including renal hyperplasia, psoriasis, chronic inflammatory diseases including rheumatoid and osteoarthritis, among others.

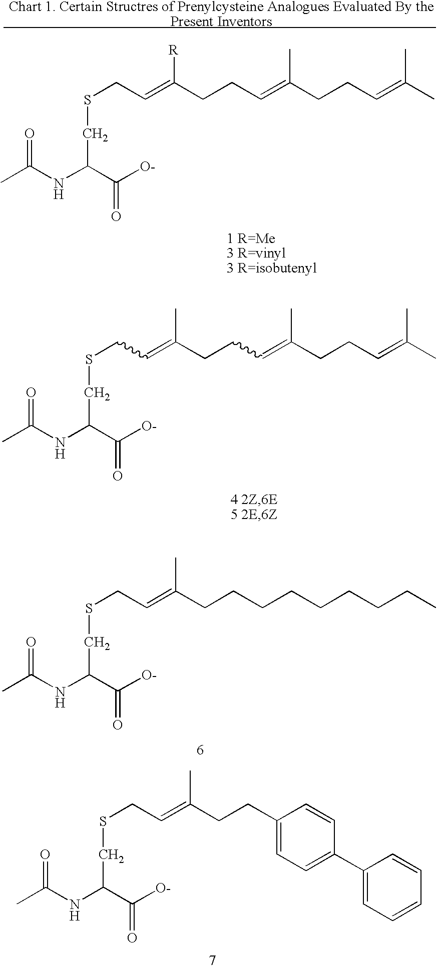

### BRIEF DESCRIPTION OF TIRE FIGURES

FIG. 1 shows the post-translational steps that transduction proteins must undergo in order to be functional in the eucaryotic cell. These proteins possess a signature carboxyl-terminal CaaX box motif, which is recognized by one of the two prenyltransferases, FTase (protein-farnesyltransferase) or GGTase I (protein-geranylgeranyltransferase I). FTase transfers the 15-carbon farnesyl moiety to the cysteine residue in certain CaaX sequences, while GGTase I transfers the 20-carbon geranylgeranyl moiety to different CaaX boxes. Ras proteins and certain other proteins are farnesylated, but the majority of naturally-occuring CaaX proteins are geranylgeranylated by GGTase I. Subsequent to prenylation, CaaX motif proteins are subjected to removal of the aaX residues by the protease RCE1, followed by SAM-dependent

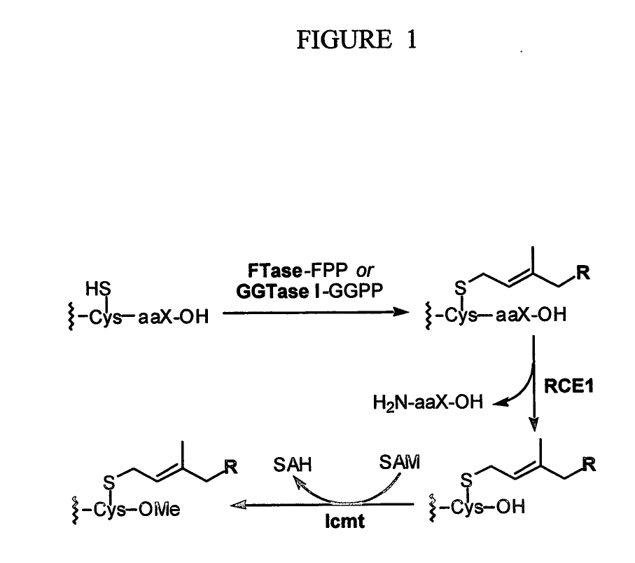

FIG. 2 shows a double reciprocal plot of inhibition of Sacharomyces cerevisiae Icmt by 3-isobutenylfarnesyl-AFC (compound 3).

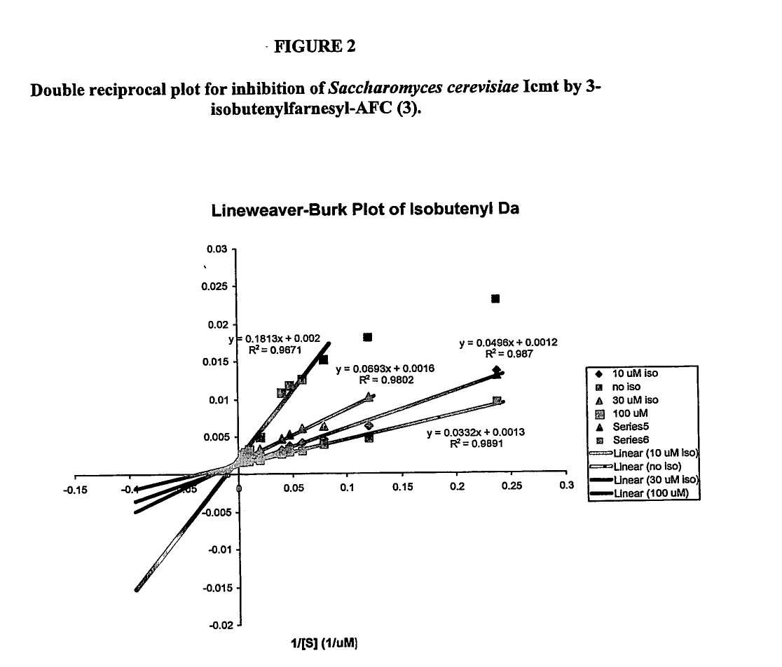

FIG. 3 shows a double receiptocal plot of inhibition of Sacharomyces cerevisiae Icmt by Biphenyl butenyl compound (compound 11).

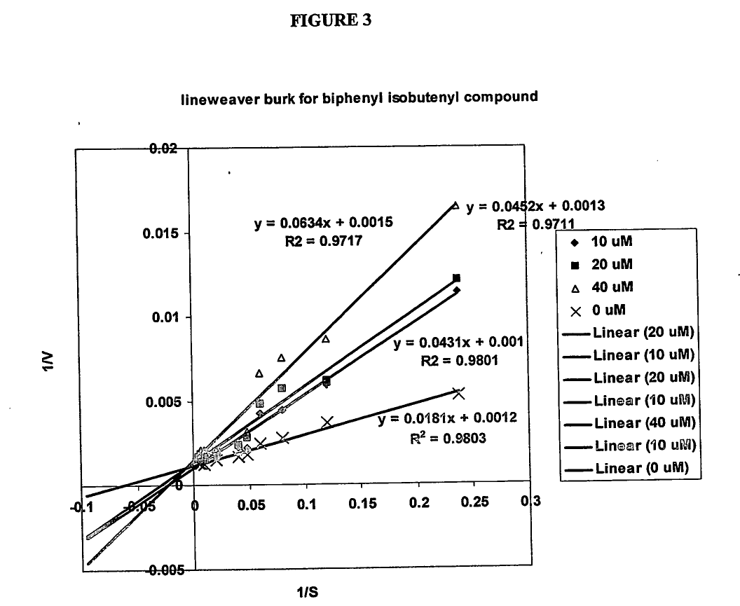

FIG. 4 shows certain additional specific preferred compounds according to the present invention.

FIG. 5 shows substrate ability of AFC and AFC analogs. Rates were determined using the vapor diffusion assay described in the experimental section. Isobutenyl—Compound 3; Biphenyl—Compound 7; AGGC—Compound 2; GG propargyl—not depicted in manuscript; structure shown below; EZ—Compound 5; AFC—Compound 3; ZE—Compound 4; Saturated—Compound 6; homoallyl—Compound 9; GG isobutenyl—Compound 12; Allyl —Compound 8; Biphenyl Isobutenyl—Compound 11; F7-isobutenyl—Compound 10; GG 7-iso —Compound 13; Isobutenyl famesol—Compound C (evaluated as a control for the importance of the N-acetyl-L-cysteine moiety).

FIG. 6 shows the inhibitory Potency of AFC analogs. Rates were determined using the vapor diffusion assay described for FIG. 4, above, in the presence of 83 μM AFC. Isobutenyl—Compound 3; Biphenyl—Compound 7; AGGC—Compound 2; GG propargyl—Compound 14; structure shown above; EZ—Compound 5; AFC—Compound 3; ZE—Compound 4; Saturated—Compound 6; homoallyl—Compound 9; GG isobutenyl—Compound 12; Allyl—Compound 8; Biphenyl Isobutenyl—Compound 11; F7-isobutenyl—Compound 10; GG 7-iso—Compound 13.

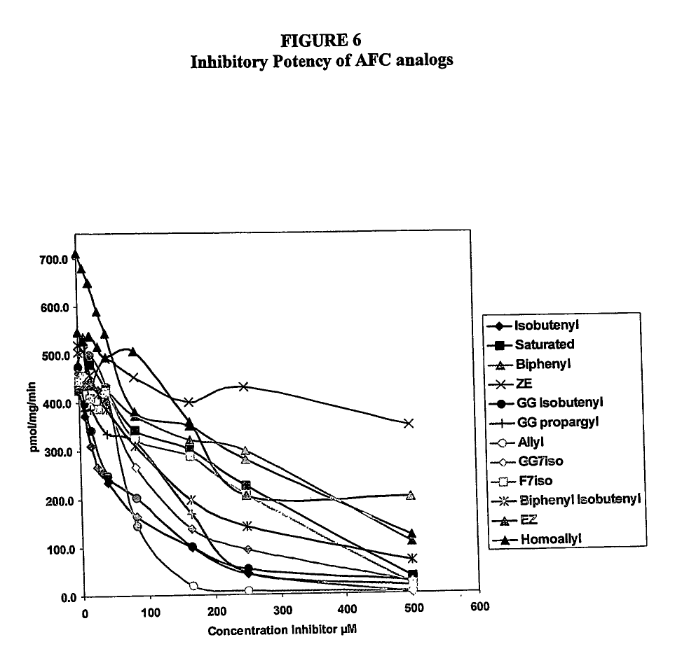

FIG. 7 shows the inhibition of GST-Ras2p methylation by 3 in the biological experimental section. Filled squares represent the base labile counts from the GST-Ras2p-containing reactions in the presence of increasing concentrations of 3. Open diamonds represent the base labile counts from the experiment in the absence of GST-Ras2p. The filled triangles represent the difference between these two data sets. The difference represents the inhibition of Icmt catalyzed GST-Ras2p methylation by 3.

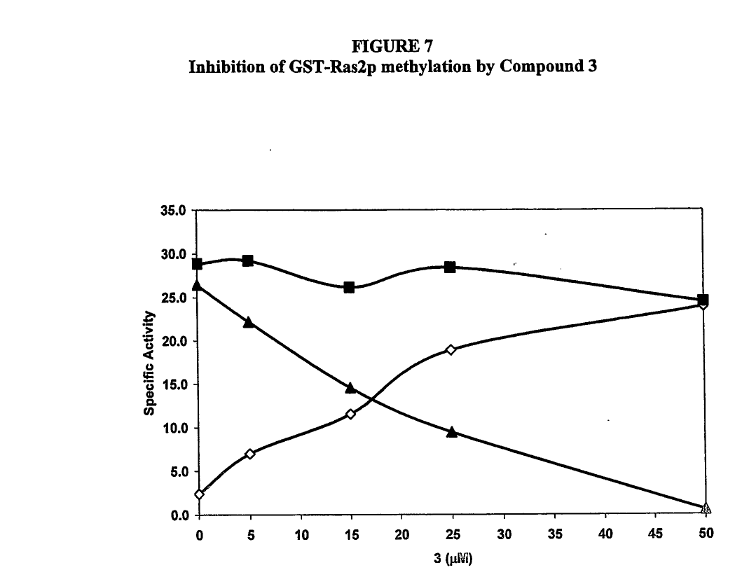

### OBJECTS OF THE INVENTION

In one aspect of the invention, an object of the present invention is to provide compounds and methods for the treatment of tumors and/or cancer in mammals.

In another aspect of the present invention, an object of the present invention is to provide pharmaceutical compositions useful for the treatment of tumors and/or cancer, hyperproliferative cell growth, restenosis following cardiovascular surgery, hyperplasia, including renal hyperplasia, psoriasis, chronic inflammatory diseases including rheumatoid and osteoarthritis, among others.

In still other aspects of the invention, objects of the present invention provide compounds and methods for the treatment of neoplasia, hyperproliferative cell growth, restenosis following cardiovascular surgery, hyperplasia, including renal hyperplasia, psoriasis, chronic inflammatory diseases including rheumatoid and osteoarthritis, among others.

In still other aspects of the present invention, objects of the invention provide methods of inhibiting isoprenylcysteine methyltransferase, an enzyme which is believed to modulate a number of disease states or conditions including neoplasia, hyperproliferative cell growth, restenosis following cardiovascular surgery, hyperplasia, including renal hyperplasia, psoriasis, chronic inflammatory diseases including rheumatoid and osteoarthritis, among others.

Any one or more of these and/or other objects of the present invention may be readily gleaned from the description of the present invention which follows.

### DESCRIPTION OF THE INVENTION

The present invention is directed to compounds of the chemical formula: 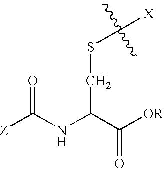
where X is selected from the group consisting of R^{a}, R^{b}, R^{c}, R^{d}, R^{e}, R^{f} and R^{g}; 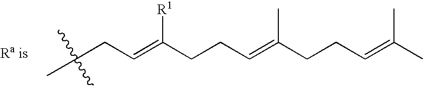
where R¹ is an isobutylene group; 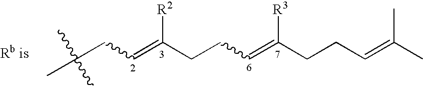
where R² and R³ are independently a C₁-C₅ linear or branched-chain alkyl or alkene group, preferably a methyl group (preferably, the double bond between carbon atoms 2 and 3 has a trans configuration when the double bond between carbon atoms 6 and 7 has a cis configuration and a cis configuration when the double bond between carbon atoms 6 and 7 has a trans configuration); 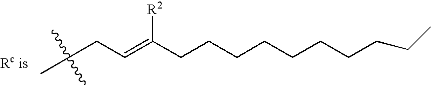
where R² is the same as above and is preferably a methyl group;
R^{d} is 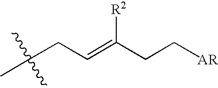
where R² is the same as above and is preferably an isobutylene group and wherein said AR group is a cyclohexyl, phenyl, naphthyl, para or ortho substituted biphenyl group, more preferably a 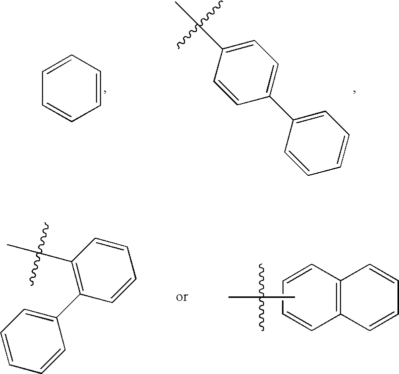
group, even more preferably 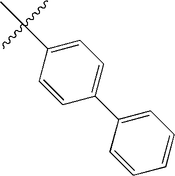
group, each group being optionally substituted with one or more halogen groups, preferably no more than three halogen groups, preferably two halogen groups, which are most preferably F;
R^{e} is 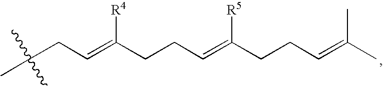
where R⁴ is a C₁-C₅ linear or branch-chained alkyl or alkene group, allyl or homoallyl, preferably allyl or homoallyl and R⁵ is a C₁-C₅ linear or branch-chained alkyl or alkene group, preferably methyl or isobutylene, more preferably isobutylene;
R^{f} is 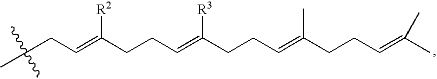
where R² and R³ are the same as is set forth above;
R^{g} is 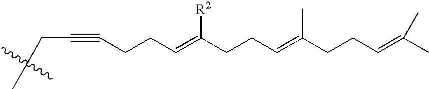
where R² is the same as is set forth above;
Z is a C₁-C₁₂ alkyl or alkylene group, or a group according to the structure 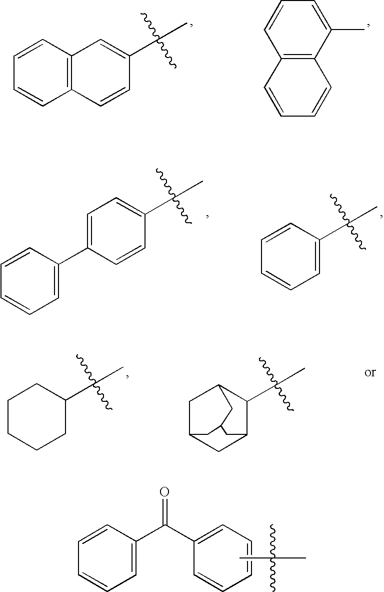
wherein each of said groups may be optionally substituted with one or more halogen groups, preferably up to three halogen groups, more preferably no more than two halogen groups,
wherein the halogen group is preferably F;
R is H or a C₁-C₁₈ alkyl group; and
pharmaceutically acceptable salts, solvates, anomers (including enantiomers) and polymorphs of the above-depicted compounds.

Other compounds according to the present invention are represented by the formula: 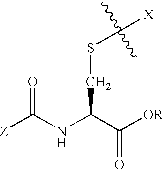
where X, Z and R are the same as is described above and pharmaceutically acceptable salts, solvates and polymorphs thereof.

In certain embodiments, a more limited group of compounds according to the present invention are represented by the formula: 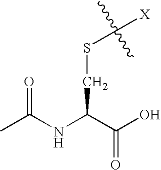
where X is R^{d} R^{e} and R^{f} as described above and pharmaceutically acceptable salts, thereof.

Pharmaceutical compositions according to the present invention comprise an effective amount of one or more of the above-depicted compounds, optionally in combination with a pharmaceutically acceptable carrier, additive or excipient.

The method of the present invention involves the use of compounds to treat neoplasia and other diseases and conditions such as hyperproliferative cell growth, restenosis following cardiovascular surgery, hyperplasia, including renal hyperplasia, psoriasis, chronic inflammatory diseases including rheumatoid and osteoarthritis, among others of animals, especially mammals, including humans encompassed by the following formula: 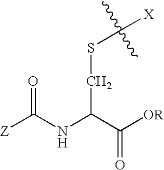
where X is selected from the group consisting of R^{a}, R^{b}, R^{c}, R^{d}, R^{e}, R^{f} and R^{g};
R^{a} is 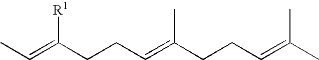
where R¹ is an isobutylene group;
R^{b} is 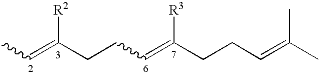
where R² and R³ are independently a C₁-C₅ linear or branched-chain alkyl or alkene group, preferably a methyl group and wherein the double bond between carbon atoms 2 and 3 has a trans configuration when the double bond between carbon atoms 6 and 7 has a cis configuration and a cis configuration when the double bond between carbon atoms 6 and 7 has a trans configuration;
R^{c} is 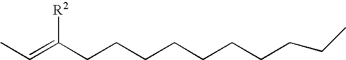
where R² is the same as above and is preferably a methyl group;
R^{d} is 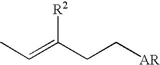
where R² is the same as above and is preferably an isobutylene group and wherein said AR group is a cyclohexyl, phenyl, naphthyl, para or ortho substituted biphenyl group, more preferably a 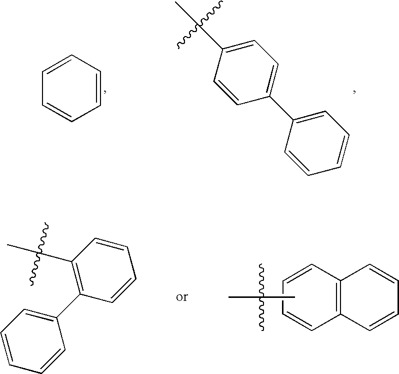
group, even more preferably
a 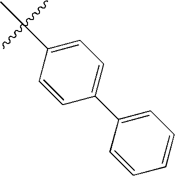
group, each group being optionally substituted with one or more halogen groups, preferably F;
R^{e} is 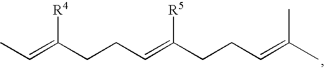
where R⁴ is a C₁-C₅ linear or branch-chained alkyl or alkene group, allyl or homoallyl, preferably allyl or homoallyl and R⁵ is a C₁-C₅ linear or branch-chained alkyl or alkene group, preferably methyl or isobutylene, more preferably isobutylene;
R^{f} is 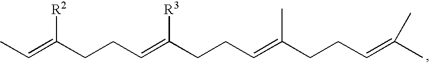
where R² and R³ are the same as is set forth above;
R⁸ is 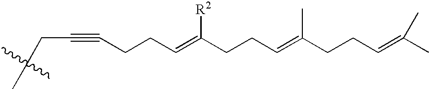
where R² is the same as is set forth above;
Z is a C₁-C₁₂ alkyl or alkylene group, or a group according to the structure 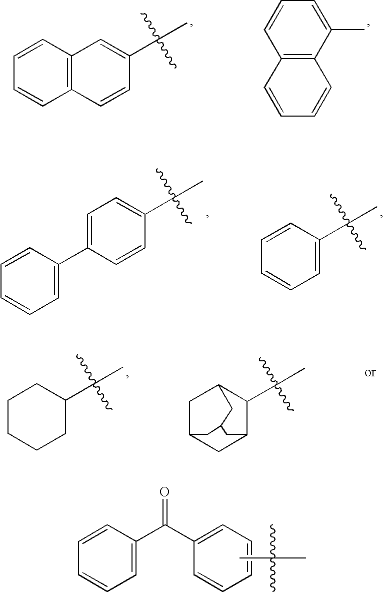
wherein each of said groups may be optionally substituted with one or more halogen groups, preferably up to three halogen groups, more preferably no more than two halogen groups,
wherein the halogen group is preferably F;
R is H or a C₁-C₁₈ allyl group; and
pharmaceutically acceptable salts, anomers, solvates and polymorphs of the above-depicted compounds.

In certain embodiments of the method aspect of the present invention the method involves the use of compounds is represented by the formula: 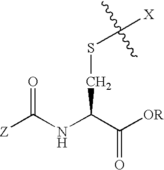
where X and Z are the same as is described above and pharmaceutically acceptable salts thereof.

In other embodiments of the method aspect of the present invention, a more limited group of compounds are used according to the formula: 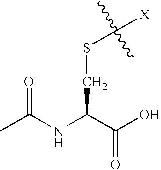
where X is R^{d} R^{e} and R^{f} as described above and pharmaceutically acceptable salts, solvates and polymorphs thereof.

The compounds of the present invention are used to treat benign and malignant neoplasia, including various cancers such as, stomach, colon, rectal, liver, pancreatic, lung, breast, cervix uteri, corpus uteri, ovary, prostate, testis, bladder, renal, brain/cns, head and neck, throat, Hodgkin's disease, non-Hodgkin's lymphoma, multiple myeloma, melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney, lymphoma, among others. Compounds according to the present invention are particularly useful in the treatment of breast cancer, including breast cancer which is of a multiple drug resistant phenotype.

A method of treating hyperproliferative cell growth, restenosis following cardiovascular surgery, hyperplasia, including renal hyperplasia, among others using one or more of the disclosed compositions are other inventive aspects of the present invention.

Further inventive aspects of the present invention relate to the use of the present compositions in the treatment of arthritis and chronic inflammatory diseases, including rheumatoid arthritis and osteoarthritis, among others.

The present invention also relates to methods for inhibiting the growth of neoplasia, including a malignant tumor or cancer comprising exposing the neoplasia to an inhibitory or therapeutically effective amount or concentration of at least one of the disclosed compounds. This method may be used therapeutically, in the treatment of neoplasia, including cancer or in comparison tests such as assays for determining the activities of related analogs as well as for determining the susceptibility of a patient's cancer to one or more of the compounds according to the present invention.

Methods for treating abnormal cell proliferation or growth of non-transformed cells, including the treatment of psoriasis, restenosis following cardiovascular surgery, hyperplasia, including renal hyperplasia, among others, chronic inflammatory diseases including rheumatoid and osteoarthritis, among others, comprising administering a therapeutically effective amount of one or more of the disclosed compounds for treating the condition or disease are also contemplated within the scope of the present invention.

The present invention also relates to a method for inhibiting isoprenylcysteine methyltransferase comprising exposing said enzyme to an effective amount of any one or more of the compounds which are set forth hereinabove.

Others aspects according to the present invention relate to a method of inhibiting isoprenyl cysteine methyltransferase enzyme in a patient in order to treat a disease or condition modulated by said enzyme comprising administering to said patient an effective amount of any one or more of the compounds compound which are set forth hereinabove. Disease states or conditions which are believed to be modulated by this enzyme include for example, neoplasia, hyperproliferative cell growth, restenosis following cardiovascular surgery, hyperplasia, including renal hyperplasia, psoriasis, chronic inflammatory diseases including rheumatoid and osteoarthritis, among others.

### DETAILED DESCRIPTION OF THE INVENTION

The following terms shall be used throughout the specification to describe the present invention.

The term “compound”, as used herein, unless otherwise indicated, refers to any specific chemical compound disclosed herein. Within its use or description in context, the term generally refers to a single compound, but in certain instances may also refer to stereoisomers (cis and/or trans, etc.) and/or optical isomers (including racemic mixtures), as well as specific enantiomers or enantiomerically enriched mixtures of disclosed compounds.

The term “patient” is used throughout the specification to describe a subject animal, such as a mammal, preferably a human, to whom treatment, including prophylactic treatment, with the compositions according to the present invention is provided. For treatment of those infections, conditions or disease states which are specific for a specific animal such as a human patient, the term patient refers to that specific animal.

The term “effective amount” is used throughout the specification to describe concentrations or amounts of compounds according to the present invention which may be used to produce an effect within context, whether that effect relates to a favorable change in the disease or condition treated, or the change is a remission, a decrease in growth or size of cancer or a tumor, a favorable physiological result, a reduction in the growth or elaboration of a microbe, or the like, depending upon the disease or condition treated.

The term “alkyl” is used throughout the specification to describe a hydrocarbon radical containing between one and five carbon units, or in the case of certain prodrug forms of the present compounds C₁-C₁₈ alkyl groups. Alkyl groups for use in the present invention include linear or branched-chain groups.

The term “neoplasia” is used to describe the pathological process that results in the formation and growth of a neoplasm, i.e., an abnormal tissue that grows by cellular proliferation more rapidly than normal tissue and continues to grow after the stimuli that initated the new growth cease. Neoplasia exhibits partial or complete lack of structural organization and functional coordination with the normal tissue, and usually form a distinct mass of tissue which may be benign (benign tumor) or malignant (carcinoma). The term “cancer” is used as a general term to describe any of various types of malignant neoplasms, most of which invade surrounding tissues, may metastasize to several sites and are likely to recur after attempted removal and to cause death of the patient unless adequately treated. As used herein, the term cancer is subsumed under the term neoplasia.

The term “hyperproliferative cell growth” is used to describe conditions of abnormal cell growth of a non-transformed cell often, of the skin, distinguishable from cancer. Examples of such conditions include, for example, skin disorders such as hyperkeratosis (including ichthyosis), keratoderma, lichen, planus and psoriasis, warts (including genital warts), blisters and any abnormal or undesired cellular proliferation.

The term “restenosis” is used to describe the recurrence of stenosis after corrective surgery on the heart, including the heart valve, or the narrowing of a structure (usually a coronary artery) following the removal or reduction of a previous narrowing of such structure.

The term “hyperplasia”, “hypertrophy” or “numerical hypertrophy” is used to describe an increase in the number of cells in a tissue or organ, excluding tumor formation and refers to all types of hyperplasia, including cystic hyperplasia, cystic hyperplasia of the breast, nodular hyperplasia of the prostate and renal hyperplasia, among numerous others.

A preferred therapeutic aspect according to the present invention relates to methods for treating neoplasia, including benign and malignant tumors and cancer in animal, especially mammalian, including human patients, comprising administering effective amounts or concentrations of one or more of the compounds according to the present invention to inhibit the growth or spread of or to actually shrink the neoplasia in the animal or human patient being treated.

Pharmaceutical compositions based upon these novel chemical compounds comprise the above-described compounds in an effective amount for the treatment of a condition or disease state such as neoplasia, including cancer, hyperproliferative cell growth, restenosis following cardiovascular surgery, hyperplasia, including renal hyperplasia, psoriasis, chronic inflammatory diseases including rheumatoid and osteoarthritis, among others or a related condition or disease as otherwise described, optionally in combination with a pharmaceutically acceptable additive, carrier or excipient.

Certain of the compounds, in pharmaceutical dosage form, may be used as prophylactic agents for preventing a disease or condition from manifesting itself. In certain pharmaceutical dosage forms, the pro-drug form of the compounds according to the present invention may be preferred.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others, which are formed at the carboxylic acid position of compounds according to the present invention.

Modifications of the active compound can affect the solubility, bioavailability and rate of metabolism of the active species, thus providing control over the delivery of the active species. Further, the modifications can affect the anticancer activity of the compound, in some cases increasing the activity over the parent compound. This can easily be assessed by preparing the derivative and testing its anticancer activity according to known methods well within the routineer's skill in the art.

The compounds of this invention may be incorporated into formulations for all routes of administration including for example, oral, topical and parenteral including intravenous, intramuscular, intraperitoneal, intrabuccal, transdermal and in suppository form, among numerous others.

Pharmaceutical compositions based upon these novel chemical compounds comprise the above-described compounds in an effective amount for treating neoplasia, cancer and other diseases and conditions which have been described herein, including psoriasis, hyperproliferative cell growth, restenosis following cardiovascular surgery, hyperplasia, including renal hyperplasia, chronic inflammatory diseases including rheumatoid and osteoarthritis, among others, optionally in combination with a pharmaceutically acceptable additive, carrier and/or excipient. One of ordinary skill in the art will recognize that a therapeutically effective amount of one of more compounds according to the present invention will vary with the infection or condition to be treated, its severity, the treatment regimen to be employed, the pharmacokinetics of the agent used, as well as the patient (animal or human) treated.

In the pharmaceutical aspect according to the present invention, the compound according to the present invention is formulated preferably in admixture with a pharmaceutically acceptable carrier. In general, it is preferable to administer the pharmaceutical composition in orally-administrable form, but a number of formulations may be administered via a parenteral, intravenous, intramuscular, transdermal, buccal, subcutaneous, suppository or other route. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity. In particular, the modification of the present compounds to render them more soluble in water or other vehicle, for example, may be easily accomplished by minor modifications (salt formulation, esterification, etc.) which are well within the ordinary skill in the art. It is also well within the routineer's skill to modify the route of administration and dosage regimen of a particular compound in order to manage the pharmacokinetics of the present compounds for maximum beneficial effect to the patient.

In certain pharmaceceutical dosage forms, the pro-drug form of the compounds may be preferred. One of ordinary skill in the art will recognize how to readily modify the present compounds to pro-drug forms to facilitate delivery of active compounds to a targeted site within the host organism or patient. The routineer also will take advantage of favorable pharmacokinetic parameters of the pro-drug forms, where applicable, in delivering the present compounds to a targeted site within the host organism or patient to maximize the intended effect of the compound.

The amount of compound included within therapeutically active formulations according to the present invention is an effective amount for treating the infection or condition. In general, a therapeutically effective amount of the present preferred compound in dosage form usually ranges from slightly less than about 0.025 mg./kg. to about 2.5 g./kg., preferably about 2.5-5 mg/kg to about 100 mg/kg of the patient or considerably more, even more preferably about 10-50 mg/kg, depending upon the compound used, the condition or infection treated and the route of administration, although exceptions to this dosage range may be contemplated by the present invention.

Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal and suppository administration, among other routes of administration.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavouring agents, preservatives, colouring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

For parenteral formulations, the carrier will usually comprise sterile water or aqueous sodium chloride solution, though other ingredients including those which aid dispersion may be included. Of course, where sterile water is to be used and maintained as sterile, the compositions and carriers must also be sterilized. Injectable suspensions may also be prepared, in which case appropriate liquid carriers, suspending agents and the like may be employed.

The present compounds may be used to treat animals, and in particular, mammals, including humans, as patients. Thus, humans, equines, canines, bovines and other animals, and in particular, mammals, suffering from tumors, and in particular, cancer, or other diseases as disclosed herein, can be treated by administering to the patient an effective amount of one or more of the compounds according to the present invention or its derivative or a pharmaceutically acceptable salt thereof optionally in a pharmaceutically acceptable carrier, additive or excipient, either alone, or in combination with other known pharmaceutical agents, depending upon the disease to be treated. This treatment can also be administered in conjunction with other conventional cancer therapies, such as radiation treatment or surgery.

The active compound is included in the pharmaceutically acceptable carrier, additive or excipient in an amount sufficient to deliver to a patient a therapeutically effective amount for the desired indication, without causing serious toxic effects in the patient treated.

The compound is conveniently administered in any suitable unit dosage form, including but not limited to one containing from less than 1 mg to a gram or more, preferably from about 1 to 3000 mg, preferably 5 to 500 mg of active ingredient per unit dosage form. An oral dose of about 25-250 mg is usually convenient.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a .number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material—of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof may also be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agents, and in certain instances depending upon the desired therapy or target, other antiprolierative agents, antirestenosis agents, antinflammatories, or other related compounds which may be used to treat disease states or conditions according to the present invention.

Solutions or suspensions used for parenteral, intradermal, subcutaneous, or topical application can include.the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers include, for example, physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art. For example, liposome formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

A wide variety of biological assays have been used and are accepted by those skilled in the art to assess anti-cancer activity of compounds. Any of these methods can be used to evaluate the activity of the compounds disclosed herein.

One common method of assessing activity is through the use of test panels of cancer cell lines. These tests evaluate the in vitro anti-cancer activity of particular compounds in cancer cell lines, and provide predictive data with respect to the use of tested compounds in vivo. Other assays include in vivo evaluations of the compound's effect on human or in an appropriate animal model, for example, using mouse tumor cells implanted into or grafted onto mice or in other appropriate animal models.

Chemical Synthesis

The compounds according to the present invention are synthesized by methods which are well known in the art. Compounds which contain the aryl, including naphthyl, or biphenyl groups as depicted above, may be readily synthesized by analogy following the well-described method of Zhou, et al., Bioorg. Med. Chem. Lett., 12, 1417-1420 (2002), relevant portions of which are incorporated by reference herein. The allyl and homoallyl containing compounds and related compounds are synthesized readily from the method of Gibbs, et al., J. Med. Chem., 1999, 42 3800-3808, relevant portions of which are incorporated by reference. The synthesis of other compounds according to the present invention occurs readily with minor modification using the well-described synthesis of Xie, et al., J. Org. Chem., 65, 8552-8563 (2000), relevant portions of which are incorporated by reference herein.

More detailed methods for the synthesis of the preferred compound 3 are given below.

The AFC analogs described below were synthesized either from famesol analogs that were previously prepared in this laboratory, or famesol analogs that were prepared using close variants of our reported procedures.^{1,2} More detailed methods for the synthesis of the most potent inhibitor 3 are given below (and depicted in Scheme S1), and a brief description of the synthesis of the other analogs is also given following this description.

Ethyl 3-(3-methylbut-2-enyl)-7,11-dimethyldodeca-2Z,6E,10-trienoate (B). Ph₃As (18.5 mg, 0.06 mmol), Pd(II) (12.8 mg, 0.033 mmol), and CuO (5.28 mg, 0.06 mmol), were charged in a round bottom flask. To this dry mixture was added 1.0 mL of NMP, and the resulting suspension was stirred at room temperature for 5 min under argon atmosphere. Then a solution of triflate A (200 mg, 0.604 mmol; prepared by the method of Gibbs et al.²), in 0.5 mL NMP was added dropwise. After 5 min at rt, tributyl(3-methyl-2-butenyl)tin (282 mg, 0.79 mmol) was added, the reaction mixture was heated to 110° C. and stirred at that temperature for 12 hr. It was the cooled, taken up in ethyl acetate (25 mL), and washed with aqueous KF (2×20 mL) and H₂O (2×20 mL). The aqueous layers were back extracted with ethyl acetate (30 mL), and the combined organic layers were dried (MgSO₄), filtered, and concentrated. Purification by flash chromatography (hexane/ethyl acetate 98:2) gave B, in a 78% yield. ¹H NMR (CDCl₃): 1.19 (t, J=14.1 Hz, 3H, CH₂CH₃), 1.51 (s, 6H, 2CH₃), 1.56-1.62 (s, 9H, 3CH₃), 1.89-2.06 (m, 8H, 4CH₂), 3.29 (d, J=7.5 Hz, 2H, CH₂), 4.07 (q, J=21.3 Hz, 2H, OCH₂), 5.03 (m, 3H, 3CH), 5.56 (s, 1H, CH); ¹³C NMR (CDCl₃): 14.27, 15.95, 17.62, 17.95, 25.62, 25.71, 26.09, 26.6, 30.92, 37.84, 39.62, 59.45, 115.18, 120.98, 123.05, 124.19, 128.59, 133.66, 135.93, 162.59, 166.56; GC-MS (Ret. Time: 8.884 min) CI (m/z): 319 (M⁺+H); Anal. Calcd. For C₂₁H₃₄O₂: C 79.20, H 10.77; found: C 79.59, H 10.93.

3-(3-methylbut-2-enyl)-7,11-dimethyldodeca-2Z,6E,10-trien-1-ol (C). A solution of the ester B (1 equivalent) in toluene (6 mL/mmol; HPLC grade dried over 4 Å sieves) was treated at −78° C. under argon with diisobutylaluminum hydride (3 equivalents; 1.0 M in toluene). After the addition the mixture was stirred for 1 h at −78° C. The reaction was quenched by adding the solution to saturated aqueous potassium sodium tartrate (40 mL), the organic phase was separated, and the aqueous phase was extracted with ethyl acetate (3×30 mL). The combined organic layers were washed with water (20 mL) and brine (20 mL) and dried by MgSO₄. Filtration and concentration followed by flash chromatography (hexane/ethyl acetate 9:1) gave C, in yields of 75-90%. This compound was characterized by proton and carbon-13 NMR, and by MS.

1-Chloro-3-(3-methylbut-2-enyl)-7,11-dimethyldodeca-2,6,10-triene (D): NCS(N-chlorosuccinimide; 2 equivalents), was dissolved in CH₂Cl₂ (distilled from CaH₂), and the resulting solution was cooled to −30° C. with a dry ice/acetonitrile bath. Dimethyl sulfide (2 equivalents) was added dropwise by a syringe, and the mixture was warmed to 0° C., maintained at that temperature for 15 min, and cooled to −30° C. To the resulting milky white suspension was added dropwise a solution of the alcohol C (1 equivalent; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. Concentration afforded the farnesyl chloride D as an oily liquid, which was used directly in the next step without purification.
N-Acetyl-S-(3-(3-methylbut-2-enyl)-7,11-dimethyldodeca-2(Z),6(E),10-trien-1-yl)-L-cysteine (Compound 3: Chloride D (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by flash column (gradient of 10-30% methanol/CH₂Cl₂) to afford compound 3 in typical yields of 40-50% based on the alcohol C. ¹H NMR (300 MHz, CDCl₃): 1.57 (s, 6H), 1.63 (s, 3H), 1.70 (two s, 6H), 2.0-2.1 (narrow m, 14H), 2.71 (narrow m, 2H), 2.9 (br, 2H), 3.16 (narrow m, 2H), 4.7 (narrow m, 1H), 4.95 (app t, 1H), 5.11 (app t, 2H), 5.24 (t, 1H), 6.45 (d, 1H), 9.3-9.7 (very br, 1H). ¹³C (75 MHz, CDCl₃) 16.42, 18.09, 18.3, 23.36, 26.11, 27.12, 30.49, 34.02, 37.42, 40.11, 122.38, 124.24, 124.74, 131.02, 132.67, 135.74, 144.307, 171.52. MS-ESI (M-H)=420. Elemental Analysis—Calculated for C₂₄H₃₈NO₃SK_{0.70}Na_{0.30}: C, 63.38; H, 8.42; Found: C, 63.28, H, 8.52. 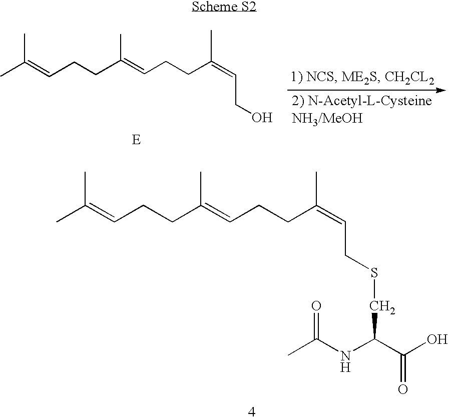
Compound 4 was synthesized from the previously described alcohol E,³ in the same manner as described above for the conversion of D to 3.
1-Chloro-3,7,11-trimethyldodeca-2(Z),6(E),10-triene: NCS(N-chlorosuccinimide; 75 mg, 0.55 mmol), was dissolved in CH₂Cl₂ (distilled from CaH₂), and the resulting solution was cooled to −30° C. with a dry ice/acetonitrile bath. Dimethyl sulfide (60 mg, 0.55 mmol) was added dropwise by a syringe, and the mixture was warmed to 0° C., maintained at that temperature for 15 min, and cooled to −30° C. To the resulting milky white suspension was added dropwise a solution of the alcohol E³ (105 mg, 0.5 mmol; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride (92 mg, 70% crude yield) was further elaborated in the next step to compound 4 without any purification. ¹H NMR (300 MHz, CDCl₃) 1.15 (s, 3H), 1.5 (s, 3H), 1.6(s, 3H), 1.7 (s, 3H), 1.9-2.0 (m, 12H), 4.2 (d, 2H), 5.0 (t, 2H), and 5.35 (t, 1H).
N-Acetyl-S-(3,7,11-trimethyldodeca-2(Z),6(E),10-trien-1-yl)-L-cysteine (Compound 4): The chloride derived from E (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by silica gel flash column chromatography (gradient of 10-30% methanol/CH₂Cl₂) to afford compound 3 in typical yields of 40-50% based on the alcohol E. ¹H NMR: (300 MHz, CDCl₃) 1.57 (s, 9H), 1.66 (s, 3H), 1.9-2.0 (m, 8H), 2.0 (s, 3H), 3.0 (d, 2H), 3.2 (d, 2H), 4.6 (q, 1H), 5.2 (t, 2H), 5.45 (t, 1H) and 6.45 (d, 1H). MS ESI (M-H)=366. 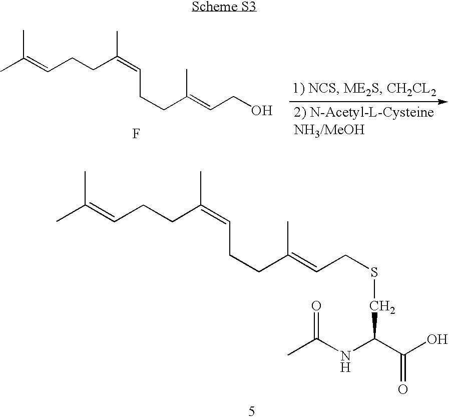
Compound 5 was synthesized from the previously described alcohol F,³ in the same manner as described above for the conversion of D to 3.
1-Chloro-3,7,11-trimethyldodeca-2(E),6(Z),10-triene: NCS(N-chlorosuccinimide; 60 mg, 0.42 mmol), was dissolved in CH₂Cl₂ (distilled from CaH₂), and the resulting solution was cooled to −30° C. with a dry ice/acetonitrile bath. Dimethyl sulfide (30 mg, 0.42 mmol) was added dropwise by a syringe, and the mixture was warmed to 0° C., maintained at that temperature for 15 min, and cooled to −30° C. To the resulting milky white suspension was added dropwise a solution of the alcohol F (80 mg, 0.38 mmol; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride (60 mg, 66% yield) was further elaborated to compound 5 without any purification. ¹H NMR (300 MHz, CDCl₃) 1.3 (s, 3H), 1.5 (s, 3H), 1.6 (s, 3H), 1.7 (s, 3H), 2.0-2.1 (m, 12H), 4.0 (d, 2H), 5.1 (t, 2H), and 5.45 (t, 1H).
N-Acetyl-S-(3,7,11-trimethyldodeca-2(E),6(Z),10-trien-1-yl)-L-cysteine (Compound 5): The chloride derived from F (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by silica gel flash column chromatography (gradient of 10-30% methanol/CH₂Cl₂) to afford compound 5 in typical yields of 40-50% based on the alcohol F. ¹H NMR: (300 MHz, CDCl₃) 1.57 (s, 9H), 1.66 (s, 3H), 1.9-2.0 (m, 8H), 2.0 (s, 3H), 3.0 (d, 2H), 3.2 (d, 2H), 4.6 (app q, 1H), 5.2 (t, 2H), 5.45 (t, 1H) and 6.45 (d, 1H). ¹³C (75 MHz, CDCl₃)16.5, 18.06, 23.33, 23.79, 26.14, 26.64, 26.99, 32.38, 40.32, 53.85, 124.7, 124.93, 131.95, 135.89, and 140.38 MS ESI (M-H)=366. 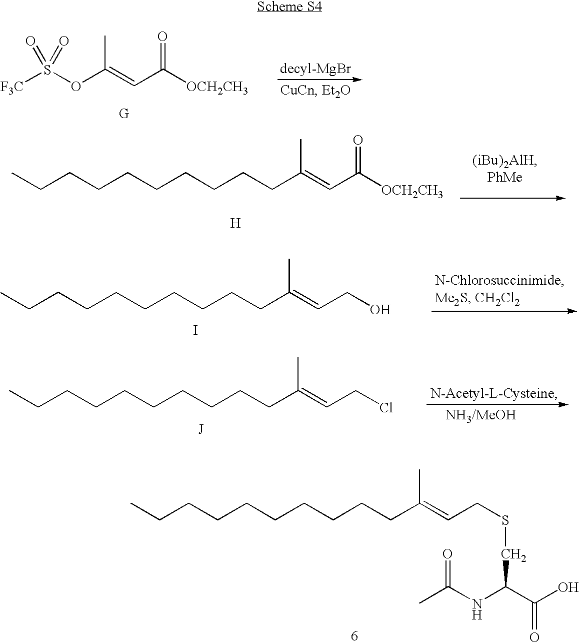
AFC analog 6 was prepared from alcohol I, the one-carbon homolog of the previously reported 3-methyldodec-2-en-1-ol.⁴ This alcohol was prepared from the previously described vinyl triflate G⁵ as illustrated in FIG. S4.
Ethyl 3-(trifluoromethylsulfonyl)-but-2E-enoate (Triflate G): Dissolve sodium ethyl

acetoacetate (1.0 mmol) in DMF and cool to 0° C. Once cool add potassium bis(trimethylsilyl)amide (KHMDS, 1.1 mmol) dropwise. After five minutes has elapsed, the 2-[N,N-bis(trifluoromethylsulfonyl)amino]-5-chloropyridine (1.2 mmol) was added. The reaction was warmed to room temperature over 12 hours. The solution is diluted with ether and the reaction was quenched with 10% aqueous citric acid solution. The aqueous layer was extracted with ether (3×15 mL). The organic layers were combined, washed with brine (30 mL), dried with MgSO₄, filtered and concentrated. The crude mixture was purified by flash chromatography (hexanes/ethyl acetate 99:1) to give triflate G in a 61% yield (161 mg). ¹H NMR (300 MHz, CDCl₃): 0.8 (t, 3H), 1.3 (s, 3H), 4.2 (q, 2H) and 5.9 (1H, s). This compound,⁵ and the stereoisomeric 2Z-triflate,⁶ have been previously reported.

Ethyl 3-methyltridec-2E-enoate (Compound H): Decyl magnesium bromide (2.4 mL of a

2.0 M soln in ether, 4.8 mmol) and CUCN (221 mg, 2.49 mmol) were suspended in anhydrous ether and cooled to −78° C. The mixture was warmed to 0° C. for 5 minutes and cooled to −78° C. The triflate G (220 mg, 0.83 mmol) was dissolved in anhydrous ether and added to the decyl magnesium bromide and CuCN solution dropwise. The mixture was stirred vigorously for 2.5 hours. The solutions was then warmed to 0° C. and quenched with a 10% aqueous ammonium chloride solution. The layers were separated, and the aqueous layer was extracted with ethyl acetate (3×10 mL). The organic layers were combined, dried (MgSO₄), filtered and concentrated. Purification by flash chromatography (19:1 hexane/ethyl acetate) gave Compound H in a yield of 75% (150 mg). ¹H NMR (300 MHz, CDCl₃): 0.8 (t, 3H), 1.25 (narrow m, 20H), 1.4 (s, 3H), 1.6 (2H), 3.6 (t, 3H), 4.2 (q, 2H) and 5.9 (s, 1H).

3-Methyl-tridec-2E-en-1-ol (Compound I): Compound H (0.19 mmol) was dissolved in anhydrous toluene (3 mL) and chilled to −78° C. DIBAL-H (0.585 mmol, 2.0 M in toluene) was added dropwise. The solution reacted for 45 minutes and was warmed slightly. The reaction was quenched with 10% aqueous sodium potassium tartrate. The layers were separated and the aqueous layer was extracted (3×20 mL) with ethyl acetate. The organic layers were combined, washed with brine (10 mL), dried, filtered and concentrated. Purification by flash chromatography (9:1 hexane/ethyl acetate) gave Compound I in yields of 85-90%. ¹H NMR (300 MHz, CDCl₃): 0.8 (t, 3H), 1.25 (narrow m, 20H), 1.5 (s, 3H), 1.8 (narrow m, 2H), 4.0 (d, 2H), and 5.4 (t, 1H).

1-Chloro-3-methyl-tridec-2-ene (Compound J): NCS(N-chlorosuccinimide; 68 mg, 0.55 mmol), was dissolved in CH₂Cl₂ (distilled from CaH₂), and the resulting solution was cooled to −30° C. with a dry ice/acetonitrile bath. Dimethyl sulfide (34 mg, 0.58) was added dropwise by a syringe, and the mixture was warmed to 0° C., maintained at that temperature for 15 min, and cooled to −30° C. To the resulting milky white suspension was added dropwise a solution of the alcohol I (1 equivalent; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride (95% crude yield, 109 mg) was concentrated and used directly in the next step without any purification. ¹H NMR (300 MHz, CDCl₃): 0.8 (t, 3H), 1.25 (narrow m, 20H), 1.5 (s, 3H), 1.8 (2H), 3.9 (d, 2H), and 5.4 (t, 1H).
N-Acetyl-S-(3-methyldodeca-2E-en-1-yl)-L-cysteine (Compound 6): Chloride J (65 mg, 0.28 mmol) and N-acetyl-L-cysteine (45 mg, 0.28 mmol) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by flash column (gradient of 10-30% Methanol in CH₂Cl₂) to afford compound 6 in a yield of 65% (64 mg). ¹H NMR: (300 MHz, CDCl₃) 0.8 (t, 3H), 1.1 (m, 18H), 1.55 (s, 3H), 2.9 (d, 2H), 3.1 (d, 2H), 4.65 (d, 1H), 5.2 (t, 1H) and 6.3 (d, 2H). MS ESI (M-H)=356. 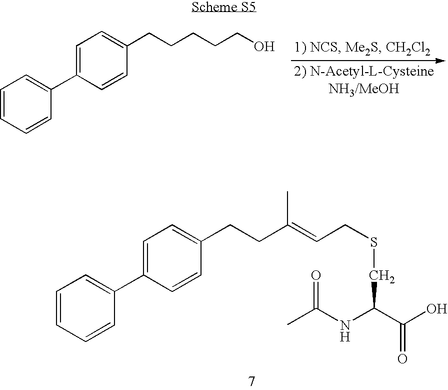
AFC analog 7 was prepared from the known alcohol K,⁷ as illustrated above.
1-Chloro-3-methyl-5-(4-phenyl)phenylpent-2E-ene: NCS(N-chlorosuccinimide; 67 mg, 0.55 mmol), was dissolved in CH₂Cl₂ (distilled from CaH₂), and the resulting solution was cooled to −30° C. with a dry ice/acetonitrile bath. Dimethyl sulfide (39 μL, 0.55 mmol) was added dropwise by a syringe, and the mixture was warmed to 0° C., maintained at that temperature for 15 min, and cooled to −30° C. To the resulting milky white suspension was added dropwise a solution of the alcohol K (107 mg, 0.42 mmol; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride (71% yield, 82 mg) was further elaborated to compound 7 without any purification. ¹H NMR (300 MHz, CDCl₃): 1.6 (s, 3H), 2.6 (m, 2H), 3.0 (m, 2H), 4.3 (d, 2H), 5.7 (t, 1H), 7.2 (t, 2H), 7.3 (t, 1H), 7.35 (d, 2H), 7.45 (d, 2H) and 7.5 (d, 2H).
N-Acetyl-S-(3-methyl-5-(4-phenyl)phenylpent-2E-en-1-yl)-L-cysteine (Compound 7): The chloride derived from K (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by silica gel flash column chromatography (gradient of 10-30% methanol/CH₂Cl₂) to afford compound 7 in typical yields of 40-50% based on the alcohol K. ¹H-NMR: (300 MHz, CDCl₃) 1.66 (s, 3H), 2.4 (t, 2H), 2.8-3.0 (m, 4H), 3.2 (m, 2H) 4.7 (m, 1H), 5.2 (t, 1H), 7.2 (t, 2H), 7.3 (t, 1H), 7.35 (d, 2H), 7.45 (d, 2H) and 7.5 (d, 2H). ¹³C (75 MHz, CDCl₃) 14.54, 18.38, 23.06, 23.53, 26.19, 29.85, 30.43, 32.0, 34.73, 39.15, 122.45, 127.34, 129.12, 133.09, 139.07, 144.41, 141.66 and 143.38. MS ESI (M-H)=450. 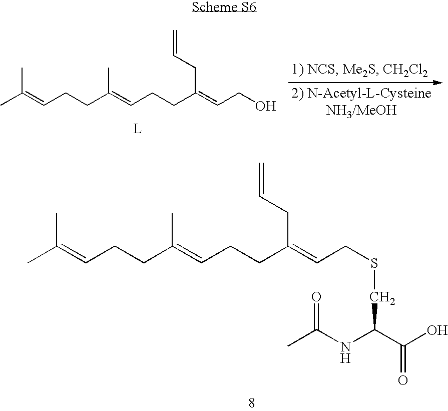
AFC analog 8 was prepared from the known alcohol L,² as illustrated above. N-Acetyl-S-(3-allyl-7,11-dimethyldodeca-2,6,10-triene-1-yl)-L-cysteine (Compound 8): ¹H NMR: (300 MHz, CDCl₃) 1.57 (s, 6H), 1.66 (s, 3H), 1.9 (q, 8H), 2.0 (s, 3H), 2.8 (d, 2H), 3.0 (d, 2H), 3.2 (d, 2H), 5.0 (t, 1H), 5.1 (t, 1H), 5.2 (t, 1H), and 5.7 (dd, 2H). MS ESI (M-H)=392. 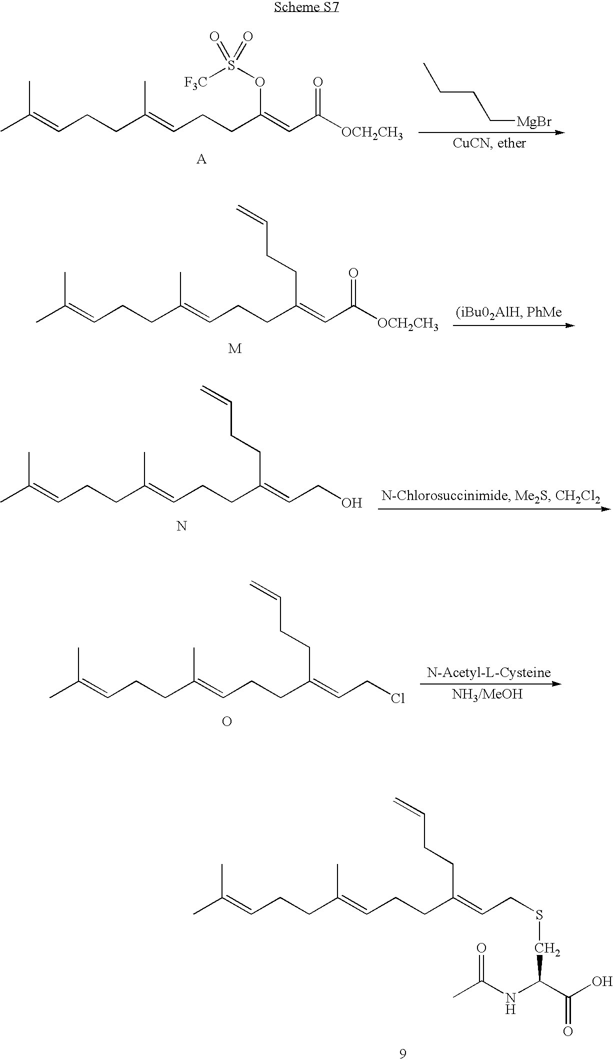
AFC analog 9 was prepared from the homoallyl alcohol N, as illustrated above. Alcohol M has been previously synthesized in our laboratory (Zahn, T. J.; PhD Dissertation, Wayne State University, 1999), and the details for its synthesis are given below.
3-(But-3-en-1-yl)-7,11-dimethyldodeca-2E,6E,10-trienoate ethyl ester: The copper cyanide(325 mg, 3.66 mmol) was suspended in ether and chilled to −78° C. The homoallyl magnesium bromide reagent (4.88 mL of a 0.5 M solution, 2.44 mmol) was added and the mixture was warmed to 0° C. for five minutes. The mixture was again chilled to −78° C. and triflate A (500 mg, 1.22 mmol) was added to the reaction slowly. After 90 minutes the reaction was warmed to 0° C. and quenched with 10% aq. ammonium chloride. The organic layer and the aqueous layers were separated and the aqueous layer was extracted three times with ether. The organic layers were combined, dried with magnesium sulfate, filtered and concentrated under reduced pressure. The product was purified using flash chromatography with 1% ethyl acetate in hexanes produced the ester M in a 84% yield (313 mg). ¹H NMR (300 MHz, CDCl₃) 1.1 (t, 3H), 1.45 (s, 3H), 1.55 (s, 3H) 1.9 (m, 6H), 2.1 (m, 6H), 2.55 (t, 3H), 4.05 (q, 2H), 4.8 (t, 1H), 4.9 (d, 2H), 5.5 (t, 1H), and 5.7 (m, 2H).
3-(But-3-en-1-yl)-7,11-dimethyldodeca-2E,6E,10-trien-1-ol: Compound M (313 mg, 1.02 mmol) was dissolved in anhydrous toluene (3 mL) and chilled to −78° C. DIBAL-H (3 mL of a 1M soln, 3.0 mmol) was added dropwise. The solution reacted for 1 hour and was warmed slightly. The reaction was quenched with 10% aqueous sodium potassium tartarate. The layers were separated and the aqueous layer was extracted (3×20 mL) with ethyl acetate. The organic layers were combined, washed with brine (10 mL), dried, filtered and concentrated. Purification was performed by flash chromatography (hexane/ethyl acetate 90:10) and gave compound N in 58% yield (150 mg). ¹H NMR (300 MHz, CDCl₃) 1.1 (t, 3H), 1.45 (s, 3H), 1.55 (s, 3H) 1.9 (m, 6H), 2.1 (m, 6H), 4.3 (d, 2H) 4.8 (t, 1H), 4.9 (d, 2H), 5.6 (t, 1H), and 5.7 (m, 1H).
1-Chloro-3-(But-3-en-1-yl)-7,11-dimethyldodeca-2E,6E,10-trienoate: NCS(N-chlorosuccinimide;58 mg, 0.41 mmol), was dissolved in CH₂Cl₂ (distilled from CaH₂), and the resulting solution was cooled to −30° C. with a dry ice/acetonitrile bath. Dimethyl sulfide (90 mg, 0.43 mmol) was added dropwise by a syringe, and the mixture was warmed to 0° C., maintained at that temperature for 15 min, and cooled to −30° C. To the resulting milky white suspension was added dropwise a solution of the alcohol N (180 mg, 0.38 mmol; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride O (80 mg, 76% yield) was further elaborated to compound 9 without any purification. ¹H NMR (300 MHz, CDCl₃) 1.1 (t, 3H), 1.45 (s, 3H), 1.55 (s, 3H) 1.9 (m, 6H), 2.1 (m, 6H), 4.2 (d, 2H) 4.8 (t, 1H), 4.9 (d, 2H), 5.5 (t, 1H), and 5.7 (m, 1H).
N-Acetyl-S-(3-(but-3-enyl)-7,11-dimethyldodeca-2Z,6E,10-trien-1-yl)-L-cysteine (Compound 9): The chloride O (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by silica gel flash column chromatography (gradient of 10-30% methanol/CH₂Cl₂) to afford compound 5 in typical yields of 40-50% based on the alcohol N. ¹H NMR: (300 MHz, CDCl₃) 1.57 (s, 6H), 1.66 (s, 3H), 1.8-2.0 (m, 12H), 3.0 (d, 2H), 3.2 (d, 2H), 5.0 (t, 1H), 5.1 (t, 1H), 5.2 (t, 1H), and 5.7 (dd, 2H). ¹³C (75 MHz, CDCl₃)16.45, 18.09, 23.51, 26.09, 27.14, 30.09, 33.14, 37.28, 40.11, 115.23, 124.2, 124.72, 131.69, 135.75, 138.66 and 143.64. MS ESI (M-H)=406. 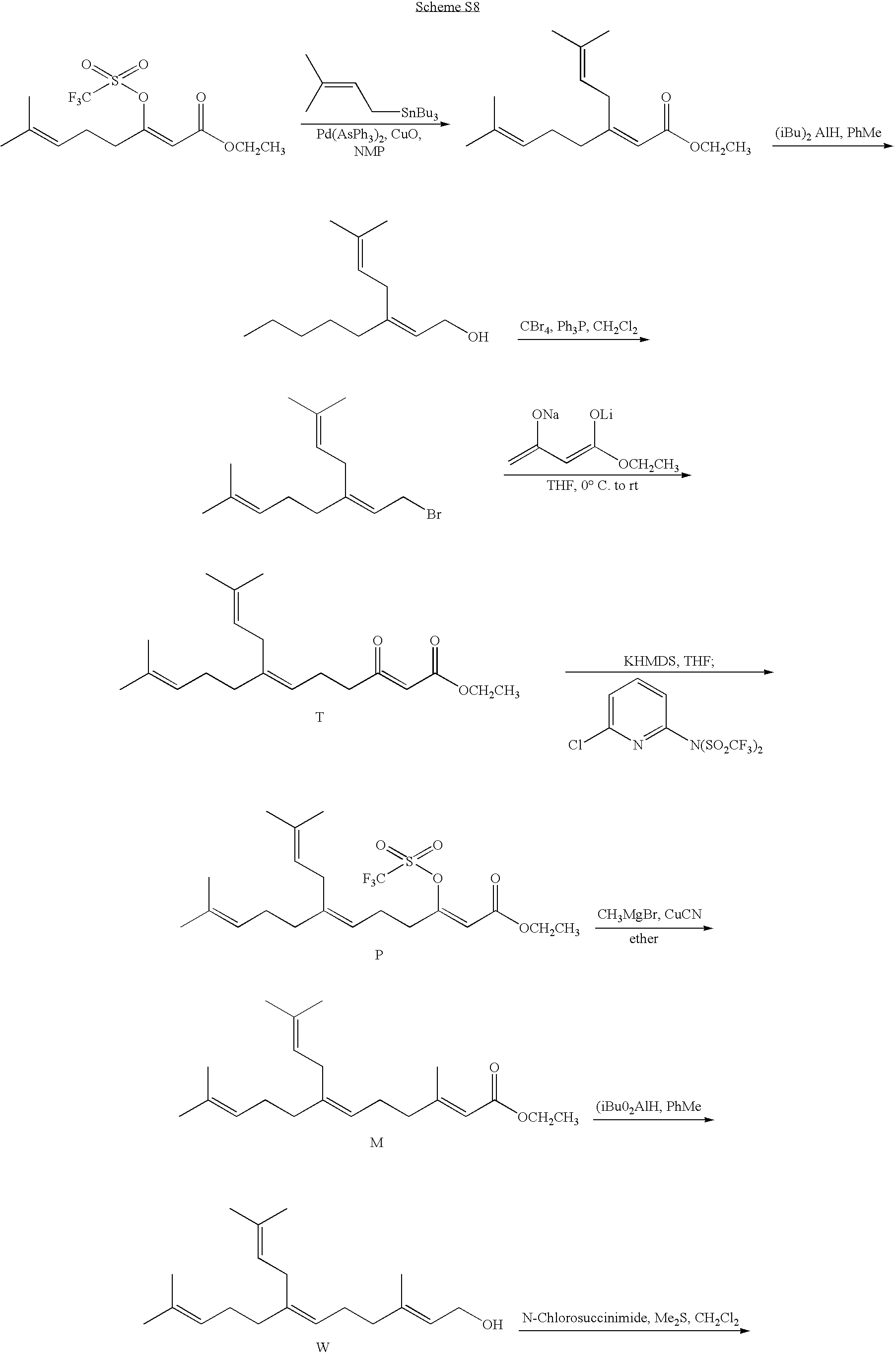 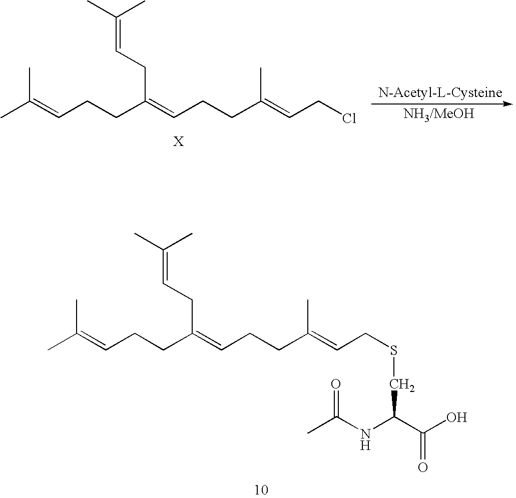
The AFC analog 10 was prepared from the corresponding chloride X, as illustrated above. Chloride N was synthesized in our laboratory from the known triflate P via the general method recently reported for the synthesis of 7-substituted famesol analogs, ⁸ and the details for its synthesis are given below.
Ethyl 3-(3-Methyl-2-butenyl)-7-methylocta-2E,6-dienoate (compound Q): Triflate P (1.8 g, 5.41 mmol; prepared by the method of Rawat. and Gibbs),⁸ CuO (430 mg, 5.4 mmol), Ph₃As (165 mg, 0.54 mmol), and bis(benzonitrile)-palladium (II) chloride (114 mg, 0.29 mmol) were placed in an argon-flushed flask and dissolved in NMP (6 mL). The mixture was immersed in an oil bath maintained at a temperature of 100-105° C., (3-methylbut-2-enyl)tributyltin (8.2 mmol) was added, and the reaction mixture was stirred for 12 h. It was the cooled, taken up in ethyl acetate (25 mL), and washed with aqueous KF (2×20 mL) and H₂O (2×20 mL). The aqueous layers were back extracted with ethyl acetate (30 mL), and the combined organic layers were dried (MgSO₄), filtered, and concentrated. Purification by flash chromatography (hexane/ethyl acetate 98:2) gave compound Q in an 89% yield (1.2 g). ¹H NMR (300 MHz, CDCl₃): 1.4 (t, 12H), 1.7 (m, 4H), 2.7 (t, 3H), 3.35 (d, 21), 4.2 (q, 2H), 5.3 (t, 2H) and 5.5 (t, 1H).
3-(3-Methyl-2-butenyl)-7-methylocta-2E,6-diene-1-ol (compound R): Compound Q (1.2 g, 4.84 mmol) was dissolved in anhydrous toluene (3 mL) and chilled to −78° C. DIBAL-H (1.88 g, 13.55 mmol) was added dropwise. The solution reacted for 1 hour and was warmed slightly. The reaction was quenched with 10% aqueous sodium potassium tartarate. The layers were separated and the aqueous layer was extracted (3×20 mL) with ethyl acetate. The organic layers were combined, washed with brine (10 mL), dried, filtered and concentrated. Purification was performed by flash chromatography (hexane/ethyl acetate 90:10) and gave compound R in 70% yield (700 mg). The structure of this compound was confirmed by ¹H NMR.
1-Bromo-3-(3-methyl-2-butenyl)-7-methylocta-2E,6-diene (compound S): A solution of the alcohol R (700 mg, 3.41 mmol), carbon tetrabromide (1.9 g, 5.8 mmols), and triphenyl phosphine (1.42 g, 4.26 mmol) was made in anhydrous dichloromethane (15 mL) and cooled to 0° C. The mixture was warmed to room temperature over an hour. The solution was concentrated and then resuspended in hexanes and filtered. It was then dried with MgSO₄ and concentrated. The product S (800 mg, 87% yield) was used directly in the next step without purification. ¹H NMR (300 MHz, CDCl₃): 1.5 (t, 12H), 1.7 (m, 4H), 2.9 (d, 2H), 4.0 (d, 2H), 5.1 (m, 3H).
Ethyl 3-Oxo-7-(3-methyl-2-but-en-yl)-11-methyldodeca-E,10-dienoate (compound T): Sodium ethyl acetoacetate (1.59 g, 10.45 mmols) was dissolved in anhydrous THF (25 mL) and cooled to 0° C. The dianion was then generated by the dropwise addition of a 2.0 M n-BuLi solution (5.2 mL, 10.45 mmols). The reaction was allowed to proceed for 30 minutes, and then bromide S (800 mg, 2.98 mmol) was added. After 45 minutes the reaction was quenched with 10% aqueous citric acid. The aqueous layer was extracted with ether (3×15 mL). The organic layers were combined, washed with brine (30 mL), dried with MgSO₄, filtered and concentrated. The compound was purified by flash chromatography (hexanes/ethyl acetate 99:1) and gave the product T in 70% yield (668 mg). ¹H NMR (300 MHz, CDCl₃): 1.2 (t, 3H), 1.6 (t, 12H), 2.1 (m, 4H), 2.3 (q, 2H), 2.5 (t, 2H), 2.7 (d, H), 3.35 (s, 2H), 4.1 (q, 2H), and 5.0-5.1 (m, 3H).
Ethyl-3-(trifluoromethylsulfonyl)-7-(but-3-methyl-2-en-1-yl)-11-methyldodeca-2E,6E,10-trienoate (Compound U): β-ketoester T (240 mg, 0.75 mmol) was dissolved in 10 mL of THF and cooled to −78° C. Potassium bis(trimethylsilyl)amide (KHMDS; 2 mL of a 0.5 M solution, 0.98 mmol) was added dropwise. After five minutes has elapsed, 2-[N,N-bis(trifluoromethylsulfonyl)amino]-5-chloropyridine (384 mg, 0.98 mmol) was added as a solid. The reaction was warmed to room temperature over 12 hours. The solution is diluted with ether and the reaction was quenched with 10% aqueous citric acid solution. The aqueous layer was extracted with ether (3×15 mL). The organic layers were combined, washed with brine (30 mL), dried with MgSO₄, filtered and concentrated. The crude mixture was purified by flash chromatography (hexanes/ethyl acetate 99:1) and gave the triflate U in 53% yield (180 mg). ¹H NMR (300 MHz, CDCl₃): 1.2 (t, 3H), 1.7 (t, 12H), 2.1 (m, 4H), 2.3 (t, 2H), 2.4 (t, 2H), 2.7 (d, 2H), 4.2 (q, 2H), and 5.0 (t, 1H), and 5.1 (t, 2H).
Ethyl 7-(But-3-methyl-2-en-1-yl)-3,11-dimethyldodeca-2E,6E,10-trienoate (Compound V): Copper (I) cyanide (104 mg, 1.17 mmol) was suspended in ether and chilled to −78° C. The methyl magnesium bromide (0.25 mL of a 3M solution, 0.78 mmol) reagent was added and the mixture was warmed to 0° C. for five minutes. The mixture was again chilled to −78° C. and triflate U was added to the reaction slowly as a solution in ether. After 90 minutes the reaction was quenched with 10% aq. ammonium chloride. The organic layer and the aqueous layers were separated and the aqueous layer was extracted three times with ether. The organic layers were combined, dried with magnesium sulfate, filtered and concentrated under reduced pressure. The crude mixture was purified using flash chromatography with 1% ethyl acetate in hexanes and product V was obtained in a 90% yield (105 mg). ¹H NMR (300 MHz, CDCl₃): 1.2 (t, 3H), 1.7 (t, 12H), 1.8 (t, 2H), 1.9 (t, 2M), 2.1 (t, 3M), 2.7 (d, 2H), 4.2 (q, 2H), and 5.0 (t, 1H), and 5.1 (t, 2H).
7-(But-3-methyl-2-en-1-yl)-3,11-dimethyldodeca-2E,6E,10-triene-ol (Compound W): Compound V (165 mg, 0.52 mmol) was dissolved in anhydrous toluene (3 mL) and chilled to −78° C. A 1M solution of DIBAL-H (2.0 M in toluene; 1.46 mL, 1.46 mmol) was added dropwise. The solution reacted for 1 hour and was warmed slightly. The reaction was quenched with 10% aqueous sodium potassium tartarate. The layers were separated and the aqueous layer was extracted (3×20 mL) with ethyl acetate. The organic layers were combined, washed with brine (10 mL), dried, filtered and concentrated. Flash column purification (Hexanes/ethyl acetate 90:10) afforded a 55% yield (80 mg) of the desired alcohol W. ¹H NMR (300 MHz, CDCl₃): 1.7 (t, 12H), 1.8 (t, 2H), 1.9 (t, 2H), 2.1 (t, 3H), 2.7 (d, 2H), 4.1 (d, 2H), and 5.0 (t, 1H), and 5.1 (t, 2H).
1-Chloro-7-(but-3-methyl-2-en-1-yl)-3,11-dimethyldodeca-2,6,10-triene (Compound x): NCS(N-chlorosuccinimide;122 mg, 0.86 mmol), was dissolved in CH₂Cl₂ (distilled from CaH₂), and the resulting solution was cooled to −30° C. with a dry ice/acetonitrile bath. Dimethyl sulfide (51 mg, 0.86 mmol) was added dropwise by a syringe, and the mixture was warmed to 0° C., maintained at that temperature for 15 min, and cooled to −30° C. To the resulting milky white suspension was added dropwise a solution of the alcohol W (1 equivalent; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride was further elaborated to compound 10 without any purification (63 mg, 74% yield). ¹H NMR (300 MHz, CDCl₃) 1.7 (t, 12H), 1.8 (t, 2H), 1.9 (t, 2H), 2.1 (t, 3H), 2.7 (d, 2H), 3.9 (d, 2H), and 5.0 (t, 1H), and 5.1 (t, 2H).
N-Acetyl-S-(7-(3-methylbut-2-enyl)-3,11-dimethyldodeca-2Z,6E,10-trien-1-yl) -L-cysteine (Compound 10): The chloride X (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by silica gel flash column chromatography (gradient of 10-30% methanol/CH₂Cl₂) to afford compound 10 in typical yields of 40-50%. ¹H NMR: (300 MHz, CDCl₃) 1.57 (s, 6H), 1.63 (s, 6H), 1.66 (s, 3H), 1.9-2.0 (q, 8H), 2.04 (s, 3H), 2.6 (d, 2H), 3.0 (d, 2H), 3.3 (d, 2H), 4.7 (t, 1H), 5.0 (t, 1H), 5.1 (t, 1H), 5.2 (t, 1H), and 6.45 (bs, 1H). MS ESI (M-H)=420. 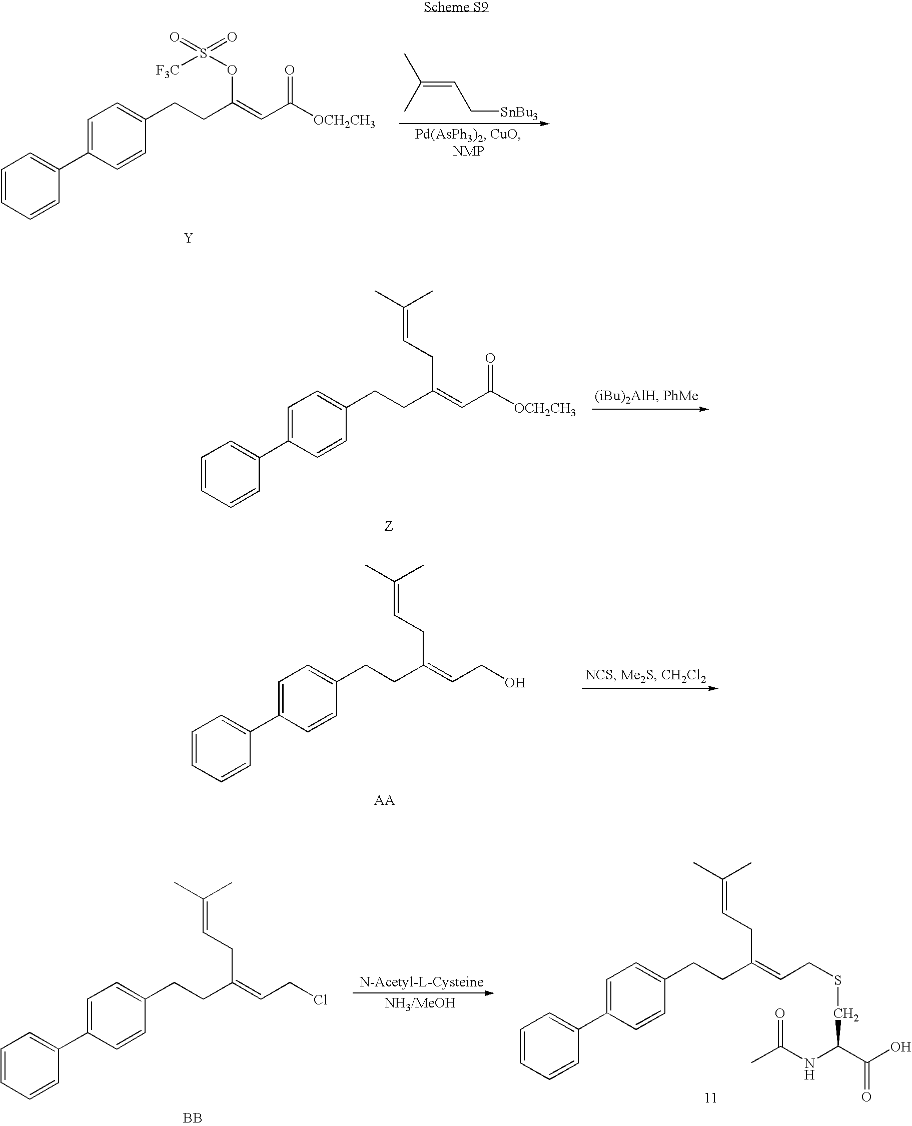
The AFC analog 11 was prepared from the known triflate y,⁷ as illustrated above and described in detail below.
Ethyl 3-(But-3-methyl-2-en-1-yl)-5-(4-phenyl)phenylpent-2E-enoate (Compound Z): Triflate Y (350 mg, 0.78 mmol), CuO (620 mg, 7.8 mmol), Ph₃As (23 mg, 0.078 mmol), and bis(benzonitrile)-palladium (II) chloride (16.5 mg, 0.0429 mmol) were placed in an argon-flushed flask and dissolved in NMP (6 mL). The mixture was immersed in an oil bath maintained at a temperature of 100-104° C., (3-methylbut-2-enyl)tributyltin (0.393 mL, 1.17 mmol) was added, and the reaction mixture was stirred for 12 h. It was the cooled, taken up in ethyl acetate (25 mL), and washed with aqueous KF (2×20 mL) and H₂O (2×20 mL). The aqueous layers were back extracted with ethyl acetate (30 mL), and the combined organic layers were dried (MgSO₄), filtered, and concentrated. Purification by flash chromatography (hexane/ethyl acetate 98:2) gave Z, in an 83% yield (230 mg). ¹H NMR (300 MHz, CDCl₃): 1.3 (t, 3H), 1.8 (t, 6H), 2.5 (t, 2H), 2.9 (t, 2H), 3.6 (d, 2H), 4.3 (q, 2H), 5.3 (t, 1H), 5.8 (t, 1H), 7.2 (t, 2H), 7.3 (t, 1H), 7.35 (d, 2H), 7.45 (d, 2H) and 7.5 (d, 2H).
3-(But-3-methyl-2-en-1-yl)-5-(4-phenyl)phenylpent-2-en-1-ol (Compound AA): Compound Z (230 mg, 0.65 mmol) was dissolved in anhydrous toluene (3 mL) and chilled to −78° C. A 1M solution of DIBAL-H (1.83 mL, 1.83 mmol) was added dropwise. The solution reacted for 1 hour and was warmed slightly. The reaction was quenched with 10% aqueous sodium potassium tartarate. The layers were separated and the aqueous layer was extracted (3×20 mL) with ethyl acetate. The organic layers were combined, washed with brine (10 mL), dried, filtered and concentrated. Purification by flash chromatography (hexane/ethyl acetate 90:10) gave alcohol AA, in a 76% yield (150 mg). ¹H NMR (300 MHz, CDCl₃): 1.8 (t, 6H), 2.5 (t, 2H), 2.9 (t, 2H), 3.6 (d, 2H), 4.1 (d, 2H), 5.3 (t, 1H), 5.8 (t, 1H), 7.2 (t, 2H), 7.3 (t, 1H), 7.35 (d, 2H), 7.45 (d, 2H) and 7.5 (d, 2H).
1-Chloro-3-(but-3-methyl-2-en-1-yl)-5-(4-phenyl)phenylpent-2E-ene (Compound BB): NCS(N-chlorosuccinimide;55 mg, 0.39 mmol), was dissolved in CH₂Cl₂ (distilled from CaH₂), and the resulting solution was cooled to −30° C. with a, dry ice/acetonitrile bath. Dimethyl sulfide (0.028 mL, 0.39 mmol) was added dropwise by a syringe, and the mixture was warmed to 0° C., maintained at that temperature for 15 min, and cooled to −30° C. To the resulting milky white suspension was added dropwise a solution of the alcohol AA (80 mg, 0.26 mmol; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride BB was further elaborated to AFC analog 11 without any purification. ¹H NMR (300 MHz, CDCl₃): 1.8 (t, 6H), 2.5 (t, 2H), 2.9 (t, 2H), 3.6 (d, 2H), 4.0 (d, 2H), 5.3 (t, 1H), 5.8 (t, 1H), 7.2 (t, 2H), 7.3 (t, 1H), 7.35 (d, 2H), 7.45 (d, 2H) and 7.5 (d, 2H).
N-Acetyl-S—(3-(3-methylbut-2-enyl)-5-(4-phenyl)phenylpent-2-en-1-yl)-L-cysteine (Compound 11): The chloride BB (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by silica gel flash column chromatography (gradient of 10-30% methanol/CH₂Cl₂) to afford compound 11 in typical yields of 40-50%. ¹H NMR: (300 MHz, CDCl₃) 1.63 (s, 6H), 2.4 (t, 2H), 2.8-3.0 (m, 6H), 3.2 (m, 2H) 4.7 (m, 11H), 5.2 (t, 1H), 5.5 (t, 1H), 7.2 (t, 2H), 7.3 (t, 11H), 7.35 (d, 2H), 7.45 (d, 2H) and 7.5 (d, 2H). MS ESI (M-H)=450. 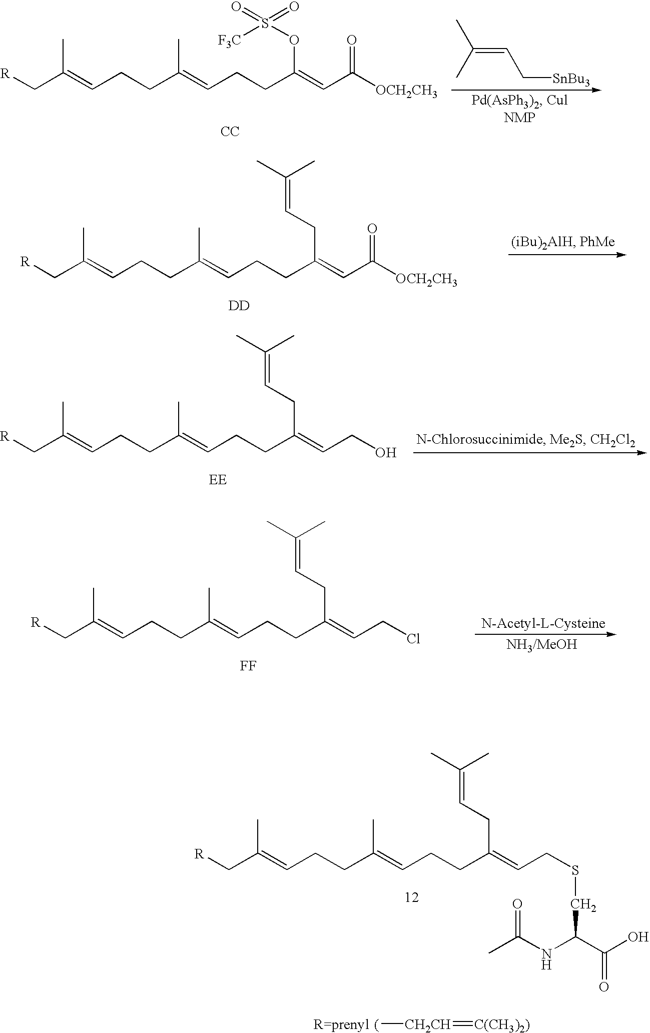
The AGGC analog 12 was synthesized from the known triflate CC⁹ using exactly the same procedures described above for the synthesis of 3, as shown in FIG. S10.
N-Acetyl-S-(3-(3-methylbut-2-enyl) 7,11,15-trimethylhexadeca-2Z,6E,10E,14-tetraen-1-yl)-L-cysteine (Compound 12): ¹H NMR: (300 MHz, CDCl₃) 1.57 (s, 6H), 1.63 (s, 6H), 1.66 (s, 3H), 1.9-2.0 (q, 12H), 2.04 (s, 3H), 2.8 (d, 2H), 3.0 (d, 2H), 3.2 (d, 2H), 4.7 (q, 1H), 5.0 (t, 1H), 5.1 (t, 2H), and 5.2 (t, 1H). ¹³C (75 MHz, CDCl₃)16.38, 18.06, 18.28, 23.41, 23.78, 26.08, 27.15, 27.30, 29.73, 30.49, 34.03, 37.45, 40.13, 50.76, 54.71, 119.78, 122.71, 124.36, 124.68, 124.79, 131.59, 132.69, 135.28, 135.61, 143.92, and 172.65. MS ESI (M-H)=488. 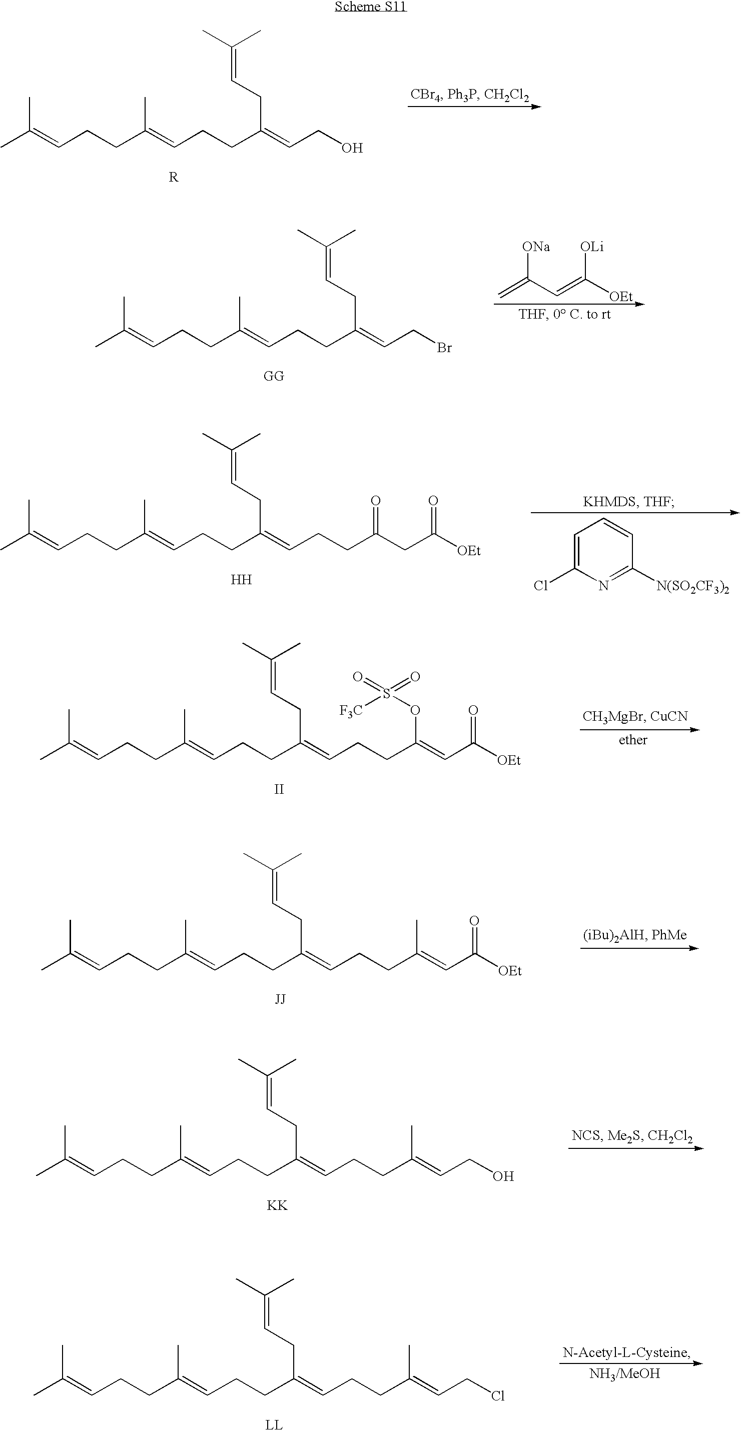 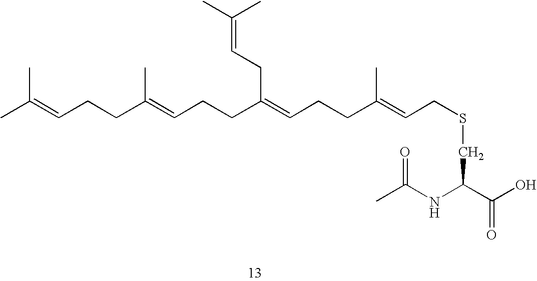
The AGGC analog 13 was synthesized from alcohol R (synthesized as previously illustrated in FIG. S1 ), via our recently described method for the synthesis of 7-substituted prenyl derivatives,⁸ as illustrated above in FIG. S11 and described below.
1-Bromo-3-(but-3-methyl-2-en-1-yl)-7,11-dimethyldodeca-2Z,6E,10-triene (compound GG): A solution of alcohol R (830 mg, 3.1 mmol), carbon tetrabromide (1.71, 5.8 mmols), and triphenyl phosphine (1.21 g, 3.8 mmol) was made in anhydrous dichloromethane and cooled to 0° C. The mixture was warmed to room temperature over an hour. The solution was concentrated and then resuspended in hexanes and filtered. It was then dried with MgSO₄ and concentrated. The product GG was produced in 90% yield (924 mg) and was further elaborated without purification. ¹H NMR (300 MHz, CDCl₃): 1.8 (s, 3H), 2.0 (t, 12H), 2.2-2.3 (m, 8H), 3.1 (d, 2H), 4.3 (d, 2H), 5.4 (m, 3H), and 5.9 (t, 1H).
Ethyl 3-Oxo-7-(but-3-methyl-2-en-1-yl)-11,15-dimethylhexadeca-6Z,10E,14-trienoate (compound HH): Sodium ethyl acetoacetate (1.43 g, 9.4 mmols) was dissolved in anhydrous THF and cooled to 0° C. The dianion was then generated by the dropwise addition of a 2.0 M n-BuLi solution (4.7 mL, 9.4 mmols). The reaction was allowed to proceed for 30 minutes, and then bromide GG (900 mg, 2.7 mmol) was added. After 45 minutes the reaction was quenched with 10% aqueous citric acid. The aqueous layer was extracted with ether (3×15 mL). The organic layers were combined, washed with brine (30 mL), dried with MgSO₄, filtered and concentrated. The compound was purified with flash chromatography (hexanes/ethyl acetate 99:1) and gave the product in 70% yield (725 mg).
3-(Trifluoromethylsulfonyl)-7-(but-3-methyl-2-en-1-yl)-11,15-dimethylhexadeca-2E,6Z,10E,14-tetraene ethyl ester (Compound II): □-ketoester HH (328 mg, 0.85 mmol) was dissolved in 10 mL THF and cooled to −78° C. Potassium bis(trimethylsilyl)amide (KHMDS,0.5 M in toluene, 2.05 mL 1.1 mmol) was added dropwise. After five minutes has elapsed, the 2-[N,N-bis(trifluoromethylsulfonyl)amino]-5-chloropyridine (433 mg, 1.1 mmol) was added. The reaction was warmed to room temperature over 12 hours. The solution is diluted with ether and the reaction was quenched with 10% aqueous citric acid solution. The aqueous layer was extracted with ether (3×15 mL). The organic layers were combined, washed with brine (30 mL), dried with MgSO₄, filtered and concentrated. The crude mixture was purified with flash chromatography (hexanes/ethyl acetate 99:1) to give the triflate II in 58% yield (256 mg). ¹H NMR (300 MHz, CDCl₃): 1.0 (t, 3H), 1.3 (t, 3H), 1.6 (t, 3H), 1.8 (t, 9H), 2.3 (t, 4H), 2.4 (t, 4H), 2.7 (d, 2H), 4.2 (q, 2H), 5.1 (t, 1H), 5.2 (t, 3H), and 5.8 (s, 1H).
Ethyl 7-(But-3-methyl-2-en-1-yl)-3,11,15-trimethylhexadeca-2E,6Z,10E,14-tetraenoate (Compound JJ): Copper (I) cyanide (100 mg, 1.15 mmol) was suspended in ether and chilled to −78° C. Then a solution of methyl magnesium bromide (3.0 M in ether; 0.25 mL, 0.76 mmol) reagent was added and the mixture was warmed to 0° C. for five minutes. The mixture was again chilled to −78° C. and triflate II (200 mg, 0.38 mmol) was added to the reaction slowly. After 90 minutes the reaction was warmed to 0° C. and quenched with 10% aq. ammonium chloride. The organic layer and the aqueous layers were separated and the aqueous layer was extracted three times with ether. The organic layers were combined, dried with magnesium sulfate, filtered and concentrated under reduced pressure. The compound was purified with flash chromatography (hexanes/ethyl acetate 99:1) and gave the product JJ in a 95% yield (140 mg). ¹H NMR (300 MHz, CDCl₃): 1.1 (t, 3H), 1.3 (t, 3H, 1.4 (t, 12H), 1.8 (m, 2H), 1.9 (m, 2H), 2.5 (d, 2H), 3.9 (q, 2H), 4.8 (t, 1H), 4.9 (t, 3H), and 5.4 (s, 1H).
7-(But-3-methyl-2-en-1-yl) 3,11,15-trimethylhexadeca-2E,6Z,10E,14-tetraen-1-ol (Compound K: Compound JJ (200 mg, 0.52 mmol) was dissolved in anhydrous toluene (3 mL) and chilled to −78° C. DIBAL-H (1.48 mL of a 1M solution, 1.48 mmol) was added dropwise. The solution reacted for 1 hour and was warmed slightly. The reaction was quenched with 10% aqueous sodium potassium tartarate. The layers were separated and the aqueous layer was extracted (3×20 mL) with ethyl acetate. The organic layers were combined, washed with brine (10 mL), dried, filtered and concentrated. The reaction mixture was purified with flash chromatography (hexanes/ethyl acetate 90:10) to give the alcohol KK in a 41% yield (70 mg). ¹H NMR (300 MHz, CDCl₃): 1.7 (t, 12H), 1.8 (m, 2H), 2.1-2.3 (m, 8H), 2.8 (d, 2H), 4.3 (d, 2H), 5.2 (t 1H), 5.3 (t, 3H), and 5.7 (t, 1H).
1-Chloro-7-(but-3-methyl-2-en-1-yl)-3,11,15-trimethylhexadeca-2,6,10,14-tetraene (Compound LL): NCS(N-chlorosuccinimide; 36 mg, 0.26 mmol), was dissolved in CH₂Cl₂ (distilled from CaH₂), and the resulting solution was cooled to −30° C. with a dry ice/acetonitrile bath. Dimethyl sulfide (18 mg, 0.3 mmol) was added dropwise by a syringe, and the mixture was warmed to 0° C., maintained at that temperature for 15 min, and cooled to −30° C. To the resulting milky white suspension was added dropwise a solution of the alcohol KK (67 mg, 0.23 mmol; dissolved in CH₂Cl₂). The suspension was warmed to 0° C. and stirred for 3 h. The ice bath was removed, and reaction mixture was warmed to room temperature and stirred for an additional 2 h. The resulting solution was washed with hexane (2×20 mL). The hexane layers were then washed with brine (2×20 mL) and dried over MgSO₄. The chloride was further elaborated to compound 13 without any purification (84% yield, 70 mg).¹H NMR (300 MHz, CDCl₃): 1.7 (t, 12H), 1.8 (m, 2H), 2.1-2.3 (m, 8H), 2.8 (d, 2H), 4.2 (d, 2H), 5.2 (t, 1H), 5.3 (t, 3H), and 5.7 (t, 1H).
N-Acetyl-S-(7-(3-methylbut-2-enyl) 3,11,15-trimethylhexadeca-2Z,6E,10E,14-tetraen-1-yl)-L-cysteine (Compound 13): The chloride LL (1 equivalent) and N-acetyl-L-cysteine (2 equivalents) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by silica gel flash column chromatography (gradient of 10-30% methanol/CH₂Cl₂) to afford compound 13 in typical yields of 40-50%. ¹H NMR: (300 MHz, CDCl₃) 1.57 (s, 6H), 1.63 (s, 6H), 1.66 (s, 3H), 1.9-2.0 (q, 12H), 2.04 (s, 3H), 2.8 (d, 2H), 3.0 (d, 2H), 3.2 (d, 2H), 4.7 (q, 1H), 5.0 (t, 1H), 5.1 (t, 2H), and 5.2 (t 1H). MS ESI (M-H)=488. 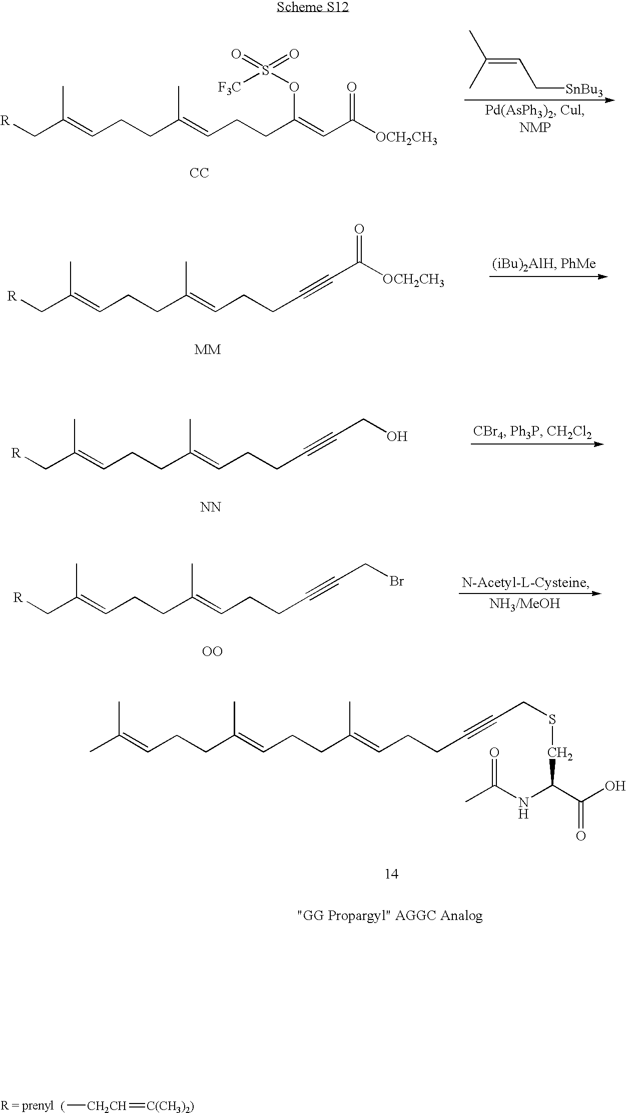
Ethyl 7,11,15-Trimethylhexadeca-6E,10E,14-trien-2-ynoate (Compound M): Triflate CC, CuI (55.3 mg, 0.29 mmol), Ph₃As (89 mg, 0.29 mmol), and bis(benzonitrile)-palladium (II) chloride (61 mg, 0.16 mmol) were placed in an argon-flushed flask and dissolved in NMP (6 mL). The mixture was immersed in an oil bath maintained at a temperature of 100-105° C., (3-methyl-but-2-en-1-yl)tributyltin (1.54 g, 1.4 mmol) was added, and the reaction mixture was stirred for 12 h. It was the cooled, taken up in ethyl acetate (25 mL), and washed with aqueous KF (2×20 mL) and H₂O (2×20 mL). The aqueous layers were back extracted with ethyl acetate (30 mL), and the combined organic layers were dried (NgSO₄), filtered, and concentrated. Purification by flash chromatography (hexane/ethyl acetate 98:2) gave 559 mg of MM, in a 61% yield %. ¹H NMR (300 MHz, CDCl₃) 1.3 (t, 3H), 1.6 (s, 9H), 1.7 (s, 3H), 2.0-2.2 (m, 12H), 2.4 (m, 4H), 4.3 (q, 2H), and 5.1-5.2 (m, 3H).
7,11,15-Trimethyl-hexadeca-6E,10E,14-trien-2-yn-1-ol (Compound NN): Ester MM (500 mg, 1.58 mmol) was dissolved in anhydrous toluene (3 mL) and chilled to −78° C. DIBAL-H (625 mg, 4.4 mmol) was added dropwise. The solution reacted for 1 hour and was warmed to 0° C. The reaction was quenched with 10% aqueous sodium potassium tartarate. The layers were separated and the aqueous layer was extracted (3×20 mL) with ethyl acetate. The organic layers were combined, washed with brine (10 mL), dried, filtered and concentrated. Purification by flash chromatography (hexane/ethyl acetate 90:10) gave 150 mg of NN, in a 29% yield %. The structure of this compound was confirmed by ¹H and ¹³C NMR
1-Bromo-7,11,15-trimethyl-hexadeca-6E,10E,14-trien-2-yne (Compound OO): A solution of propargyl alcohol NN (150 mg, 0.55 mmol), carbon tetrabromide (310 mg, 0.94 mmols), and triphenyl phosphine (180 mg, 0.69 mmol) was made in anhydrous dichloromethane and cooled to 0° C. The mixture was warmed to room temperature over an hour. The solution was concentrated and then resuspended in hexanes and filtered. It was then dried with MgSO₄ and concentrated. The crude bromide OO (65% crude, 120 mg) was taken directly to the next step without purification. ¹H NMR (300 MHz, CDCl₃) 1.3 (t, 3H), 1.6 (s, 9H), 1.7 (s, 3H), 2.0-2.2 (m, 12H), 4.1 (d, 2H), and 5.1-5.2 (m, 3H).
N-Acetyl-S-(7,11,15-trimethyl-hexadeca-6E,10E,14-trien-2-yne)-L-cysteine (Compound 14) Bromide OO (120 mg, 0.36 mmol) and N-acetyl-L-cysteine (65 mg, 0.40) were dissolved in 7.0 N NH₃/MeOH (10 mL/mmol chloride), stirred at 0° C. for 1 h and then at 20° C. for 1 h. The resulting mixture was concentrated by rotary evaporation. The crude compound was taken up in MeOH/CH₂Cl₂ and directly purified by flash column (gradient of 10-30% Methanol in CH₂Cl₂) to afford compound 14. ¹H NMR: (300 MHz, CDCl₃) 1.3 (t, 3H), 1.6 (s, 9H), 1.7 (s, 3H), 2.0-2.2 (m, 12H), 3.5 (m, 2H), 4.8 (m, 1H), and 5.1-5.2 (m, 3H) 5.5 (s, 3H), and 7.2 (broad s, 1H). ¹³C (75 MHz, CDCl₃): 16.4, 16.5, 18.1, 19.8, 21.1, 23.4, 26.1, 27.0, 27.2, 27.9, 31.3, 35.1, 40.1, 53.9, 75.4, 84.9, 122.9, 122.5, 124.7, 131.6, 135.4, 137.1, and 172.0. 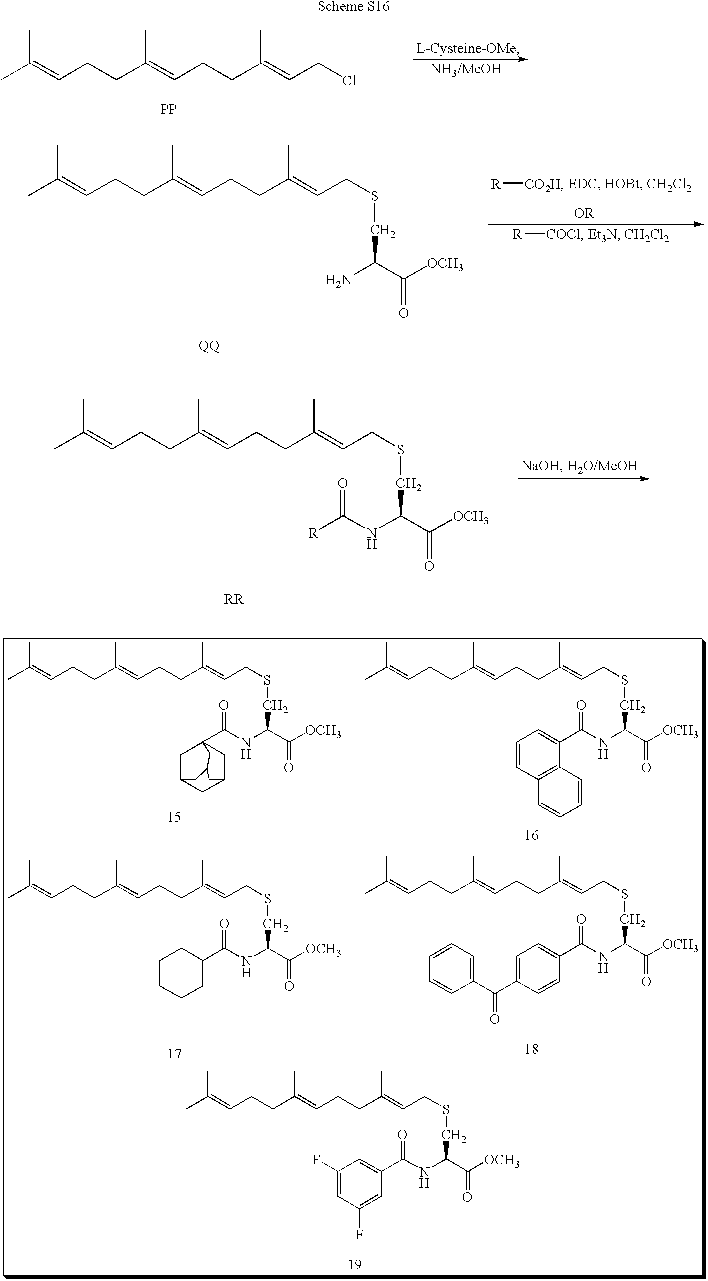
Synthesis of the amine modified AFC analogs (15-19) is performed as is shown above. The Cysteine methyl ester hydrochloride salt is dissolved in a commercially available solution of methanol containing 7N ammonia at 0° C. The farnesyl chloride (PP) is added slowly and reaction is maintained at 0° C. for one hour. The reaction is allowed to warm to room temperature and is monitored for an additional one to two hours. The farnesylcysteine product (QQ) is purified by flash chromatography with a methanol and dichloromethane mobile phase. Acylation of the farnesylated cysteine's free amine can be done either by standard peptide coupling methodology utilizing the carboxylic acid for of the of the group to be added, or by treatment of the free amine compound with an acid chloride under basic conditions. Saponification of the methyl ester (RR) is carried out by dissolving the ester in methanol at 0° C. Aqueous sodium hydroxide is added dropwise to the solution and the solution is warmed to room temperature over the course of two hours. The following analytical data were obtained for compounds synthesized according to the above described scheme.
N-1-adamantanyl-S-(3,7,11-trimethyldodeca-2(E),6(E),10-trien-1-yl)-L-cysteine (Compound 15) ¹H NMR (300 MHz, CDCl₃): 1.5 (s, 3H), 1.6 (s, 6H), 1.7 (s, 3H), 2.1 (m, 10H), 2.2 (m, 8H), 2.8 (bs, 2H), 3.1 (d, 2H), 3.9 (bs, 1H), 5.2 (t, 2H), 5.4, (t, 1H), and 6.8 (bs, 1H).
N-1-naphtyl-S-(3,7,11-trimethyldodeca-2(E),6(E),10-trien-1-yl)-L-cysteine (Compound 16) ¹H NMR (300 MHz, CDCl₃): 1.5 (s, 3H), 1.6 (s, 6H), 1.7 (s, 3H), 2.1-2.2 (m, 10H). 3.2 (m, 2H), 5.1 (t, 1H), 5.4 (t, 2H), 6.6 (d, 1H), 7.8 (m, 3H), and 8.2 (m, 4H).
N-cyclohexyl-S-(3,7,11-trimethyldodeca-2(E),6(E),10-trien-1-yl)-L-cysteine (Compound 17) ¹H NMR (300 MHz, CDCl₃): 1.5 (s, 3H), 1.6 (s, 6H), 1.7 (m,10H), 2.1 (m, 10H), 3.2 (d, 2H), 4.4 (bs, 1H), 5.2 (t, 2H), 5.4 (t, 1H), and 6.9 (d, 1H).
N-benzoyl-(4-benzoyl)-S-(3,7,11-trimethyldodeca-2(E),6(E),10-trien-1-yl)-L-cysteine (Compound 18) ¹H NMR (300 MHz, CDCl₃): 1.5 (s, 3H), 1.6 (s, 6H), 1.7 (m,10H), 2.1-2.2 (m, 10H), 3.3 (d, 2H), 5.2 (t, 2H), 5.4 (t, 1H), 7.8 (m, 2H), 7.9 (t, 1H), 8.0 (m, 4H) and 8.2 (m, 2H).
N-3,5 difluorobenzoyl-S-(3,7,11-trimethyldodeca-2(E),6(E),10-trien-1-yl)-L-cysteine (Compound 19) ¹H NMR (300 MHz, CDCl₃): 1.5 (s, 3H), 1.6 (s, 6H), 1.7 (m,10H), 2.1 (m, 8H), 2.2 (d, 2H), 3.2 (m, 2H), 4.6 (d, 1H), 5.3-5.4 (m, 3H), 7.8 (m, 2H), 6.8 (t,1H), 7.4 (bs, 2H) and 8.0 (bs, 1H).

Other compounds according to the present invention may be readily synthesized by analogy following the procedures which are described in detail above, in combination with the synthetic disclosures which may be found in Zhou, et al., Bioorg. Med. Chem. Lett., 12, 1417-1420 (2002); Gibbs, et al., J. Med. Chem., 1999, 42 3800-3808; and Xie, et al., J. Org. Chem., 65, 8552-8563 (2000).

### EXAMPLES/BIOLOGICAL ACTIVITY

Previous studies in this laboratory have focused on the isoprenoid substrate specificity of FTase. We have discovered analogues that are effective alternative substrates, and compounds that are potent inhibitors. The fact that subtle changes in the structure of the analogue leads to a complete reversal in biological activity was intriguing, and suggests that a related pattern might emerge from a similar study of Icmt. In the initial study, six isoprenoid-modified analogues (2-7, Chart 1, below) of the minimal Icmt substrate AFC 1 were prepared and evaluated as substrates and as inhibitors of the enzyme. These isoprenoid moieties were chosen in part because the corresponding FPP analogues are alternative substrates for FTase, and can thus be readily incorporated into peptides and proteins. The assays were performed using overexpressed, reconstituted recombinant Saccharomyces cerevisiae Icmt.

Preliminary evaluation of analogues 2-7 demonstrated that they possess a wide variation in their ability to act as Icmt substrates (Table 1). These results demonstrate that the prenyl moiety is a major binding determinant in the interaction between Icmt and its prenylated protein substrates. Note in particular that all six of these modified prenyl moieties afford FPP analogues that can be effectively incorporated into proteins by FTase, yet when incorporated into AFC, they vary widely in their ability to act as substrates.

Analogues 2-7 were then evaluated as potential inhibitors of Icmt (Chart 2, below). Their ability to act as inhibitors varied as widely as their substrate behavior, with some compounds exhibiting no inhibitory ability. However, the 3-isobutenyl compound 3 was a low micromolar inhibitor of AFC methylation, Several other compounds were less potent, but still effective Icmt inhibitors. The most potent inhibitor, 3, was evaluated further to determine its mode of action. It was confirmed that, as expected, it is an AFC(protein)—competitive inhibitor of Icmt, with a Ki value of 21.3 μM.

The ability of 3 to inhibit Icmt lead us to synthesis several analogues in an attempt to enhance its inhibitory potency. The 3-allyl and 3-homoallyl substitutents represent a modest modification of the structure of 3, while analogue 10 was synthesized to examine the effect of the movement of the isobutenyl moiety to a different location on the isoprenoid chain. In view of the enhanced substrate ability of the model geranylgeranyl substrate AGGC (11), we therefore also prepared the AGGC analogues of 3 and 10, compounds 12 and 13. Unfortunately, of these compounds was as potent as the lead inhibitor 3. Neither was the analogue 14; however, note that this compound which possesses very little character of the parent farnesyl moiety is still a moderately effective inhibitor of Icmt.

In summary, we have initiated the examination of the isoprenoid substrate specificity of the potential anti-cancer drug target Icmt. This study has demonstrated that the substrate specificities of FTase and Icmt are quite different. This implies that the unnatural prenylation of Ras or another protein with, for example, the 3-isobutenylfarnesyl moiety would lead to a protein that could not be methylated, and in fact could be an inhibitor of the methylation of other proteins by Icmt. Furthermore, we have discovered a novel lead compound for the further development of substrate-based inhibitors of Icmt.

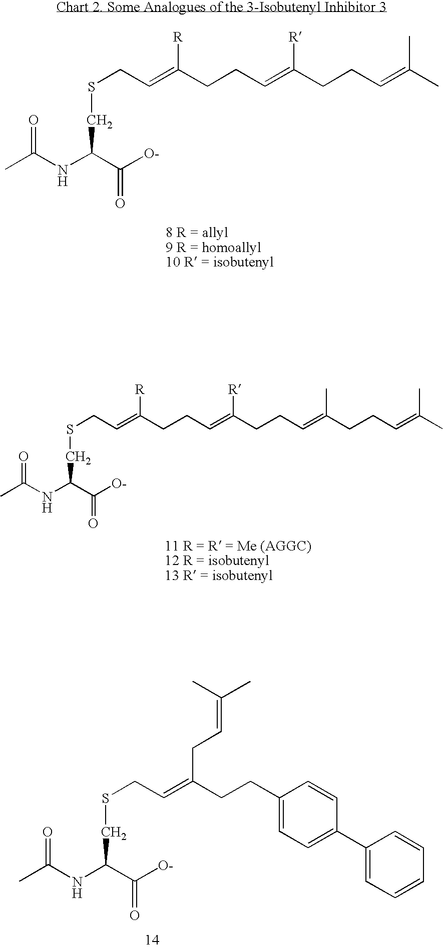

TABLE 1
Evaluation of AFC Analogues as Substrates for and Inhibitors
of Saccharomyces cerevisiae Icmt.

| | | | Icmt | |
|---|---|---|---|---|
| | | Icmt | IC₅₀ | |
| | Prenyl substituent | kᵣₑₗ^{a} | (μM)^{b} | FTase Kₘ (nM)^{c} |
| 1 | farnesyl | 100 | nd | 300 |
| 3 | 3-isobutenylfarnesyl | 5.9 | nd | 156 |
| 4 | 2E,6Z-farnesyl | 62.0 | 42 | 880 |
| 5 | 2Z,6E-farnesyl | 19.1 | 200 | 136 |
| 6 | saturated analogue | 24.2 | >500 | 225 |
| 7 | p-biphenyl analogue | 10.7 | 280 | nd |
| 8 | 3-allylfarnesyl | 18.6 | 350 | 800 |
| 9 | 3-homoallylfarnesyl | 35.4 | 125 | 119^{d} |
| 10 | 7-isobutenylfarnesyl | 35.0 | 170 | nd |
| 14 | 3-isobutenyl-p-biphenyl | 0.70 | 250 | nd |
| | analogue | | 50 | nd |
| 12 | 3-isobutenylgeranylgeranyl | 50.0 | 130 | nd |
| 13 | 7-isobutenylgeranylgeranyl | 16.3 | 130 | nd |

LEGEND
^{a}Relative velocity (compared to AFC) at 125 μM. Details presented in the supporting information section.
^{b}Details presented in the Supporting Information section.
^{b}Previously published data from references x, y and z for the corresponding FPP analogues.
^{d}IC₅₀ value for 3-allylFPP; Kₘ value was not determined.

Further Biological Results
Yeast Strains and Media—Plasmid-bearing strains were created by transformation of the indicated plasmid into SM1188, which does not express Ste14p, using the method of Elble with the following modification; DTT was added to a final concentration of 50 mM to increase the transformation efficiency. All strains were grown at 30° C. on synthetic complete solid media without uracil (SC-URA). The SM1188 strain was kindly provided by S. Michaelis (Johns Hopkins Medical Institute).

Cloning of His-Ste14p—To construct pCHm3 the 117 bp BamHI fragment from pSM937, which contains a triply iterated myc sequence tag, was cloned into the BamHI site of pSM703. The oligonucleotide 5′-CGTAGAATTCATGCATCATCATCATCATCATCATCATCATCATGGCCCGGGG AATCTC-3′ and its complement were annealed to one another, resulting in a 52 bp EcoRI-SmaI fragment, which contains a 10 histidine tag. This was cloned into the EcoRI and SmaI sites of pCHm3 to give pCHH10 m3N. A 734 bp EagI-SacII fragment, which contained the gene STE14, was amplified from pSM187 using the primers 5′-ATAAGAATCGGCCGATGCACCAAGATTTCAAGAAG-3′ and 5′-GCATCCCCGCGGTTATATAAAAGGTATTCCGACACCAACC-3′. This was cloned into the EagI and SacII sites of pCHH10 m3N to give pCHH10 m3N-STE14. This plasmid encodes Ste14p with a 10 histidine tag followed by a 3 myc epitope repeat at the N-terminus under the constitutive control of the phosphoglycerate kinase (PGK) promoter. All plasmids were sequenced bidirectionally to confirm their DNA sequence. The plasmids pSM187, pSM703, and pSM937 were kindly provided by S. Michaelis (John Hopkins Medical Institute).

Productions of Glutatione-S-Transferase (GST)-Ras2p fusion protein—A 1 kb BamHI-EagI fragment, which contained the gene RAS2, was amplified from pSM1696 using the primers 5′-CGCGGATCCTATGCCTTTGAACAAGTCG-3′ and 5′-CAACATAATATTCAATTGCCGGCATTCTTAT-3′. This was cloned into the BamHI and EagI sites of pET42-b(+) (Novagen) to give pET42-b(+)-GST-Ras2. A 1.8 kb EcoRI-EagI fragment, which contained the gene GST-RAS2, was amplified from pET42-b(+)-GST-Ras2 using the primers 5′-CCGGAATTCATGTCCCCTATAC-3′ and 5′CAACATAATATTCAATTGCCGGCATTCTTAT-3′. This was cloned into the EcoRI and EagI sites of pSM703 to give pSM703-GST-Ras2. This plasmid encodes GST tagged Ras2p. Transformation into the Ste14p lacking strain SM1188 gives the CH2735 strain. This strain produces GST-Ras2p that has been isoprenylated and proteolyzed but not methylated. All plasmids were sequenced bidirectionally to confirm their DNA sequence. The plasmids pSM187, pSM703, pSM937, and pSM1696 were kindly provided by S. Michaelis (John Hopkins Medical Institute).

Isolation of Membrane Fraction from Yeast Cells—Mid-log phase yeast cells (2.0-3.0 OD₆₀₀/mL), grown in SC-URA, media, were harvested by centrifugation at 3500×g for 10 minutes at 4° C., washed with 10 mM NaN₃, and resuspended in lysis buffer (0.3 M sorbitol, 10 mM Tris-HCl pH 7.5, 0.1 M NaCl, 5 mM MgCl₂, 1% aprotinin, 10 μg/mL leupeptin, 10 μg/in L pepstatin A, 10 μg/mL chymostatin, 10 μg/mL bestatin, 1 mM dithiothreitol, and 2 mM AEBSF) to a final concentration of 800 OD₆₀₀/mL. After a 15 min incubation on ice, the cells were frozen and thawed twice in liquid N₂. The cells were then lysed by passing the mixture twice through a French press. The resultant mixture was centrifuged at 500×g to remove whole cells and other particulate. The supernatant was then treated with 50 U/mL micrococcal nuclease followed by centrifugation at 150,000×g in a Beckman L5 50B centrifuge (45 Ti rotor) for 90 minutes at 4° C., to pellet the membrane fraction. The pellet was resuspended in lysis buffer containing 10% glycerol, aliquoted, and stored at −80° C. Membrane protein concentration was determined using Coomassie Plus Protein Assay Reagent (Pierce Biotechnology) according to the manufacturers instructions, and compared to a BSA standard curve prepared by the same procedure. Protein samples were analyzed by immunoblot analysis and in vitro vapor diffusion methyltransferase assay as described elsewhere.

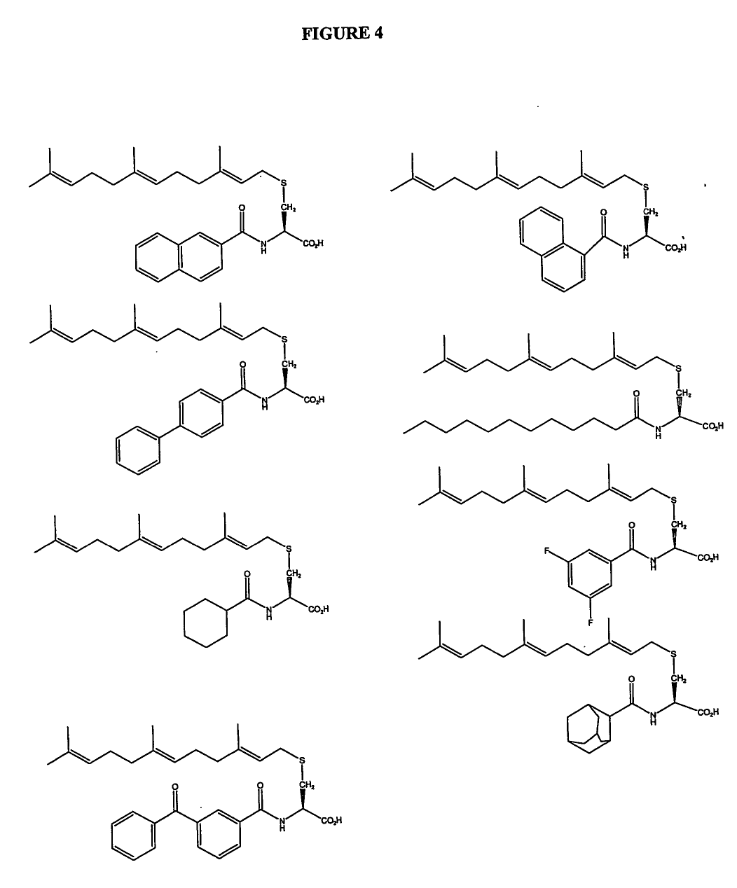

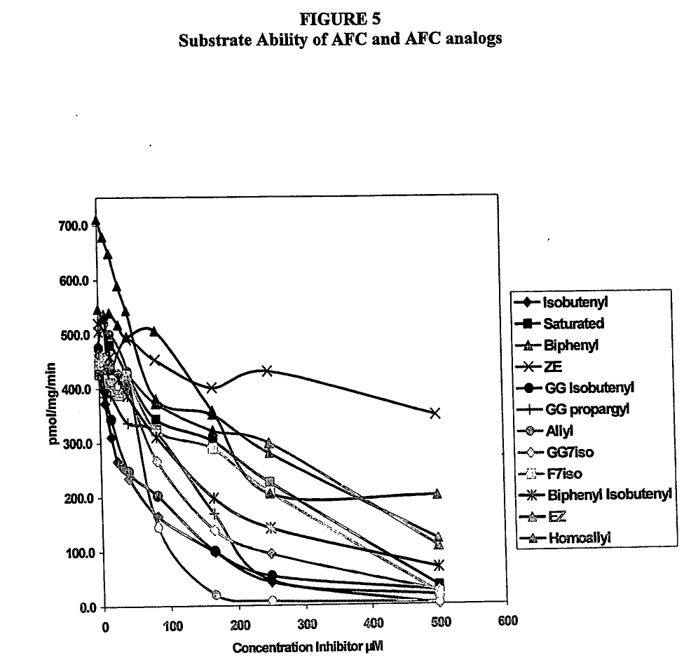

In vitro vapor diffusion methyltransferase assay—Methyltrasferase assays were performed in a total volume of 60 μL and a final Tris-HCl concentration of 100 mM, pH 7.5. All reactions contained 20 μM S-adenosyl-[¹⁴C-methyl]-methionine (SAM) and 5 μg of membrane protein from the His-Ste14p overexpressing strain CH2704. Substrate curves were generated by varying the amount of compound in each reaction. Inhibition curves were generated by varying the amount of compound in each reaction while in the presence of 33 μM N-acetyl-S-farneslylcysteine (AFC). K_{I} curves were generated by varying the amount of AFC in the presence of a constant concentration of 3-isobutenyl farnesylcysteine. The 60 μL reactions were incubated at 30° C. for 30 minutes. The reaction was stopped with the addition of 50 μL of 1M NaOH/1% SDS. 100 μL of this mixture is then spotted on folded filter paper (5.5 cm×1.5 cm) and lodged in the neck of a scintillation vial containing 10 mL of scintillation fluid. Hydroxide ion forms a tetrahedral intermediate with the newly formed ¹⁴C-methyl ester on the methyl acceptor. This intermediate then eliminates ¹⁴C-methanol, which diffuses into the scintillation fluid below. The filters were pulled out after 2-3 hours, the vials were shaken well and counted in a liquid scintillation analyzer. The results of these assays for the compounds of the present invention are set forth in FIGS. 4 and 5.

Inhibition of GST-Ras2p methylation by compound 3—The crude GST-Ras2p membrane fraction (250 μg was incubated with His-Ste14p (5 μg and [¹⁴C]S-adenosylmethionine ([¹⁴C]SAM) in the presence of increasing concentrations of compound 3. This analysis was also carried out using 250 μg of a strain lacking both Ste14p and GST-Ras2p, CH2714, to determine the background substrate activity of compound 3 at each concentration point.

Immunoblot Analysis—Gel samples were heated to 65° C. for 15 min and resolved by 12% SDS-PAGE. Proteins were transferred to pure nitrocellulose (0.2 μm; Schleicher & Schuell BioScience GmbH) at 400 mA for 1 hour. The filter was blocked with 20% milk in phosphate-buffered saline with Tween (PBST; 137 mM NaCl, 2.7 mM KCl, 4 mM Na₂HPO₄, 1.8 mM KH₂PO₄, and 0.05% Tween-20, pH 7.4) for 12-16 h at 4° C. The filter was then incubated with the primary antibody (1:1000α-Ste14, 1:2000α-His, or 1:10,000α-myc) dissolved in 5% milk in PBST for 3 h at room temperature. Following 3 washes with PBST, the filter was incubated with the secondary antibody (1:2000 goat α-mouse HRP or 1:10,000 goat α-rabbit HRP) for 1 h at room temperature. After 3 washes with PBST, the filter was visualized by chemiluminescence (Super Signal West Pico Chemiluminescent Substrate; Pierce Biochemical).

### REFERENCES

(1) Gibbs, R A.; Krishnan, U.; Dolence, J. M.; Poulter, C. D. A Stereoselective Palladium/Copper-Catalyzed Route to Isoprenoids: Synthesis and Biological Evaluation of 13-Methylidenefarnesyl Diphosphate. J. Org. Chem. 1995, 60, 7821-7829.
(2) Gibbs, B. S.; Zahn, T. J.; Mu, Y. Q.; Sebolt-Leopold, J.; Gibbs, R. A. Novel Farnesol and Geranylgeraniol Analogues: A Potential New Class of Anticancer Agents Directed against Protein Prenylation. J. Med. Chem. 1999, 42, 3800-3808.
(3) Xie, H.; Shao, Y.; Becker, J. M.; Naider, F.; Gibbs, R. A. Synthesis and Biological Evaluation of the Geometric Farnesylated Analogues of the a-Factor Mating Peptide of Saccharomyces cerevisiae. J. Org. Chem. 2000, 65, 8552-8563.
(4) McGeady, P.; Kuroda, S.; Shimizu, K; Takai, Y.; Gelb, M. H. The Farnesyl Group of H-Ras Facilitates the Activation of a Soluble Upstream Activator of Mitogen-activated Protein Kinase. J. Biol. Chem. 1995, 270, 26347-26351.
(5) Saulnier, M. G.; Kadow, J. F.; Tun, M. M.; Langley, D. R.; Vyas, D. M. Chemoselective Synthesis of Allyltrimethylsilanes by Cross-Coupling of Vinyl Triflates with Tris((trimethylsilyl)methyl)aluminum Catalyzed by Palladium(0). J. Am. Chem. Soc. 1989, 111, 8320-8321.
(6) Crisp, G. T.; Meyer, A. G. Palladium-Catalyzed, Carbonylative, Intramolecular Coupling of Hydroxy Vinyl Triflates. Synthesis of Substituted a,b-Butenolides. J. Org. Chem. 1992, 57, 6972-6975.
(7) Zhou, C.; Shao, Y.; Gibbs, R— A. Aromatic Farnesyl Diphosphate Analogues: Vinyl Triflate-Mediated Synthesis and Preliminary Evaluation. Bioorg. Med. Chem. Lett. 2002, 12, 1417-1420.
(8) Rawat, D. S.; Gibbs, R A. Synthesis of 7-Substituted Farnesyl Diphosphate Analogues. Org. Lett. 2002, 4, 3027-3030.
(9) Mu, Y. Q.; Eubanks, L. M.; Poulter, C. D.; Gibbs, R A. Coupling of Isoprenoid Triflates with Organoboron Nucleophiles: Synthesis and Biological Evaluation of Geranylgeranyl Diphosphate Analogues. Bioorg. Med. Chem. 2002, 10, 1207-1219.
(10) Hrycyna, C. A.; Wait, S. J.; Backlund, P. S.; Michaelis, S. Yeast STE14 methyltransferase, expressed as TrpE-STE14 fusion protein in Escherichia coli, for in vitro carboxylmethylation of prenylated polypeptides. Methods Enzymol. 1995, 250, 251-266.

1. A compound according to the formula: 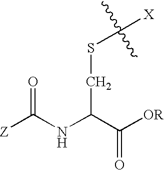
where X is selected from the group consisting of R^{a}, R^{b}, R^{c}, R^{d}, R^{e}, R^{f} and R^{g};
R^{a} is 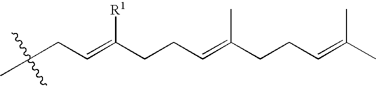
where R¹ is an isobutylene group;
R^{b} is 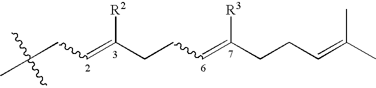
where R² and R³ are independently a C₁-C₅ linear or branched-chain alkyl or alkene group;
R^{c} is 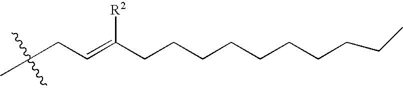
where R² is the same as above;
R^{d} is 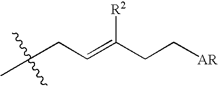
where R² is the same as above and wherein said AR group is a phenyl, naphthyl, para or ortho substituted biphenyl group;
R^{e} is 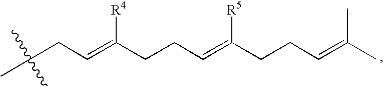
where R⁴ is a C₁-C₅ linear or branch-chained alkyl or alkene group, allyl or homoallyl group and R⁵ is a C₁-C₅ linear or branch-chained alkyl or alkene group;
R^{f} is 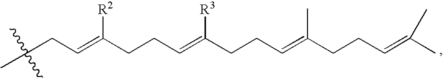
where R² and R³ are the same as is set forth above,
R^{g} is 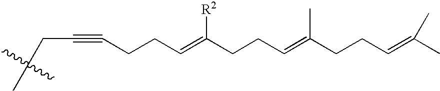
where R² is the same as is set forth above;
Z is a C₁-C₁₂ alkyl or alkylene group, or a group according to the structure 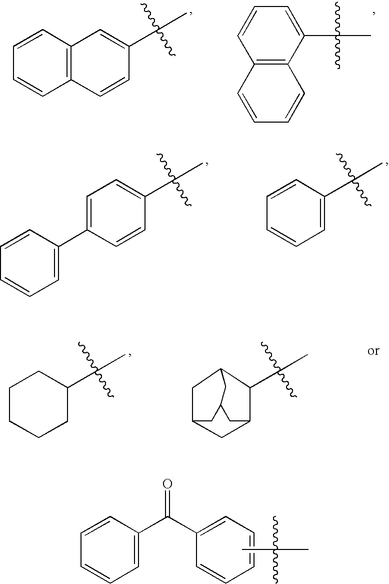
wherein each of said groups may be optionally substituted with one or more halogen groups;
R is H or a C₁-C₁₈ alkyl group; and
pharmaceutically acceptable salts, anomers, solvates and polymorphs, thereof.

2. The compound according to claim 1 wherein said formula is:

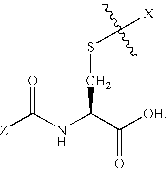

3. The compound according to claim 2 wherein X is R^{d}, R^{e} or R^{f}.

4. The compound according to claim 1 wherein R² or R⁴ is isobutenyl.

5. The compound according to claim 1 where X is R^{a}.

6. The compound according to claim 1 where X is R^{b}.

7. The compound according to claim 1 where X is R^{c}.

8. The compound according to claim 1 where X is R^{d}.

9. The compound according to claim 1 where X is R^{e}.

10. The compound according to claim 1 where X is R^{f}.

11. The compound according to claim 1 where X is R^{g}.

12. The compound according to claim 1 where R¹, R², R³, R⁴ or R⁵ is an isobutenyl group.

13. The compound according to claim 1 wherein Z is CH3.

14. The compound according to claim 1 wherein Z is a group according to the structure: 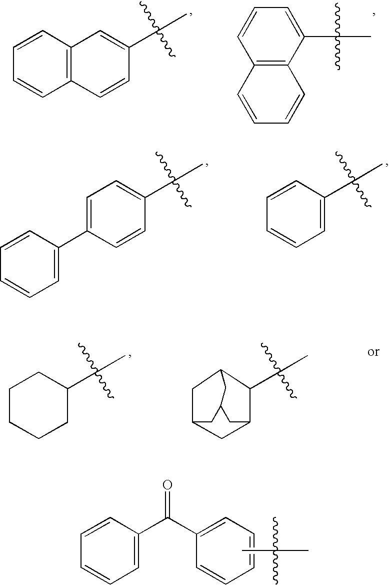
wherein each of said groups is optionally substituted with one or two fluorine groups.

15. The compound according to claim 1 wherein Z is a biphenyl group.

16. The compound according to claim 1 wherein R is H.

17. A pharmaceutically acceptable salt of the compound according to claim 14.

18. The compound according to claim 1
wherein AR is 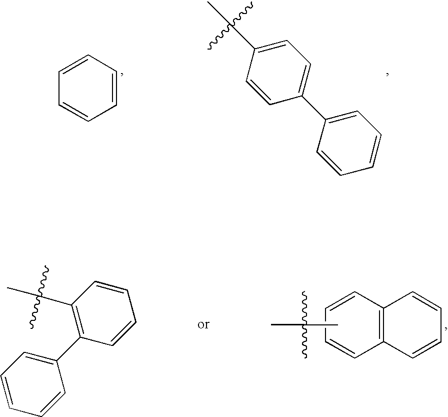
optionally substituted with 1 or 2 fluorine groups.

19. The compound according to claim 18 wherein AR is

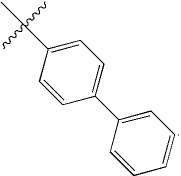

20. The compound according to claim 1 wherein R is a C₁-C₁₈ alkyl group.

21. The compound according to claim 1 wherein R² or R⁴ is isobutenyl.

22. A pharmaceutical composition comprising an effective amount of a compound according to claim 1 optionally in combination with a pharmaceutically acceptable carrier, additive or excipient.

23. A method for treating neoplasia in a patient in need thereof comprising administering to said patient an effective amount of a compound according to claim 1.

24. The method according to claim 23 wherein said neoplasia is a tumor.

25. (canceled)

26. A method for treating a patient in need thereof for a disease or condition selected from the group consisting of hyperproliferative cell growth, restenosis following cardiovascular surgery, hyperplasia and chronic inflammatory diseases comprising administering to said patient suffering from said disease an effective amount of a compound according to claim 1.

27. The method according to claim 26 wherein said hyperproliferative cell growth disease or condition is hyperkeratosis, keratoderma, lichen, planus, psoriasis, warts or blisters.

28. The method according to claim 27 wherein said hyperkeratosis is ichthyosis.

29. The method according to claim 26 wherein said hyproliferative cell growth disease or condition is psoriasis.

30. The method according to claim 27 wherein said warts are genital warts.

31. The method according to claim 26 wherein said hyperplasia is cystic hyperplasia, nodular hyperplasia of the prostate or renal hyperplasia.

32. The method according to claim 31 wherein said cystic hyperplasia is cystic hyperplasia of the breast.

33. A method for treating chronic inflammatory disease comprising administering to a patient in need of therapy an effective amount of a compound according claim 1.

34. The method according to claim 33 wherein said chronic inflammatory disease is rheumatoid arthritis or osteoarthritis.

35. A method of inhibiting isoprenylcysteine methyltransferase enzyme comprising exposing said enzyme to an effective amount of a compound according to claim 1.

36. A method of inhibiting isoprenyl cysteine methyltransferase enzyme in a patient in order to treat a disease or condition modulated by said enzyme comprising administering to said patient an effective amount of a compound according to claim 1.

37-38. (canceled)

39. The method according to claim 23 wherein said neoplasia is a cancer of the stomach, colon, rectal, liver, pancreatic, lung, breast, cervix uteri, corpus uteri, ovary, prostate, testis, bladder, renal, brain/cns, head and neck, throat, Hodgkin's disease, non-Hodgkin's lymphoma, multiple myeloma, melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma or kidney.
